(19) United States
(12) Patent Application Publication
(10) Pub. No.: US 2026/0308638 A1
(43) Pub. Date: Oct. 8, 2026

(54) CRISPR/CAS12F ENZYME AND SYSTEM

(71) Applicant: China Agricultural University, Beijing (CN)

(72) Inventors: Jinsheng LAI, Beijing (CN); Yingsi ZHOU, Beijing (CN); Jinjie ZHU, Beijing (CN); Fei YI, Beijing (CN); Xiangbo ZHANG, Beijing (CN); Haiming ZHAO, Beijing (CN); Weibin SONG, Beijing (CN)

(73) Assignee: China Agricultural University, Beijing (CN)

(21) Appl. No.: 19/712,077

(22) Filed: Jun. 18, 2026

Related U.S. Application Data
(63) Continuation of application No. 17/289,204, filed on Oct. 29, 2019, now Pat. No. 12,692,517.

(30) Foreign Application Priority Data
Oct. 29, 2018 (CN) 201811266209.7

(51) Int. Cl. C12N 15/90; C12N 9/22; C12N 15/11; C12N 15/62

(52) U.S. Cl. CPC C12N 15/902; C12N 9/22; C12N 15/111; C12N 15/62; C12N 2310/20

(57) ABSTRACT

A Cas effector protein, a fusion protein with the Cas effector protein, and a nucleic acid molecule encoding the same are provided. Also provided are a compound and a composition for nucleic acid editing (e.g., gene or genome editing) with the protein or the nucleic acid molecule, and a method for nucleic acid editing (e.g., gene or genome editing) using the protein.

### CROSS REFERENCE TO THE RELATED APPLICATION

This application is a continuation application of U.S. patent application Ser. No. 17/289,204, filed on Apr. 27, 2021. U.S. patent application Ser. No. 17/289,204 is the national phase entry of International Application No. PCT/CN2019/113996, filed on Oct. 29, 2019, which is based upon and claims priority to Chinese Patent Application No. 201811266209.7, filed on Oct. 29, 2018, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBSDSF012-POA-CONT-PKG_SequenceListing.xml, created on Jun. 18, 2026, and is 90,743 bytes in size.

### TECHNICAL FIELD

The present invention relates to the field of nucleic acid editing, in particular to the technical field of clustered regularly interspaced short palindromic repeats (CRISPR).

Specifically, the present invention relates to Cas effector proteins, fusion proteins containing such proteins, and nucleic acid molecules encoding them. The present invention also relates to complexes and compositions for nucleic acid editing (for example, gene or genome editing), which comprise the proteins or fusion proteins of the present invention, or nucleic acid molecules encoding them. The present invention also relates to a method for nucleic acid editing (for example, gene or genome editing), using that comprising the proteins or fusion proteins of the present invention.

### BACKGROUND

CRISPR/Cas technology is a widely used gene editing technology. It uses RNA guidance to specifically bind to target sequences on the genome and cut DNA to produce double-strand breaks and uses biological non-homologous end joining or homologous recombination for site-directed gene editing.

The CRISPR/Cas9 system is the most commonly used type II CRISPR system. It recognizes the PAM motif of 3′-NGG and cuts the target sequence with blunt ends. The CRISPR/Cas Type V system is a type of CRISPR system newly discovered in the past two years. It has a 5′-TTN motif and cuts the target sequence with sticky ends, such as Cpf1, C2c1, CasX, and CasY. However, the currently existing different CRISPR/Cas have different advantages and disadvantages. For example, Cas9, C2c1 and CasX all require two RNAs for guide RNA, while Cpf1 only requires one guide RNA and can be used for multiple gene editing. CasX has a size of 980 amino acids, while the common Cas9, C2c1, CasY and Cpf1 are usually around 1300 amino acids in size. In addition, the PAM sequences of Cas9, Cpf1, CasX, and CasY are more complex and diverse, and C2c1 recognizes the strict 5′-TTN, so that its target site is easier to be predicted than other systems, thereby reducing potential off-target effects.

In a word, given that the currently available CRISPR/Cas systems are limited by some shortcomings, the development of a more robust new CRISPR/Cas system with good performance in many aspects is of great significance to the development of biotechnology.

### SUMMARY

After a lot of experiments and repeated explorations, the inventor of the present invention has unexpectedly discovered a new type of RNA-guided endonuclease. Based on this discovery, the present inventor has developed a new CRISPR/Cas system and a gene editing method based on the system.

### Cas Effector Protein

Therefore, in the first aspect, the present invention provides a protein having an amino acid sequence as shown in SEQ ID NO: 1, or having an amino acid sequence having at least 90%, at least 91%, at least 92%, at least 93%, at least 94%, at least 95%, at least 96%, at least 97%, at least 98%, or at least 99% sequence identity compared to SEQ ID NO: 1, the amino acid sequence substantially retains the biological function of SEQ ID NO:1.

In certain embodiments, the present invention provides a protein having an amino acid sequence as shown in SEQ ID NO:1 or an ortholog, homolog or variant thereof; wherein the ortholog, homolog or variant has at least 90%, at least 91%, at least 92%, at least 93%, at least 94%, at least 95%, at least 96%, at least 97%, at least 98%, or at least 99% sequence identity compared to SEQ ID NO:1, and substantially retains the biological function of SEQ ID NO:1.

In the present invention, the biological function of the above sequence includes, but is not limited to, the activity of binding to guide RNA, endonuclease activity, and the activity of binding to and cleaving a specific site of the target sequence guided by the guide RNA.

In certain embodiments, the protein is an effector protein in the CRISPR/Cas system.

In certain embodiments, the protein of the present invention has an amino acid sequence as shown in SEQ ID NO:1.

### Derived Protein

The protein of the present invention can be subjected to derivatization, for example, linked to another molecule (for example, another polypeptide or protein). Generally, the derivatization of the protein (for example, labeling) will not adversely affect the desired activity of the protein (for example, the activity of binding to the guide RNA, endonuclease activity, the activity of binding to and cleaving a specific site of the target sequence guided by the guide RNA). Therefore, the protein of the present invention is also intended to include such derivatized forms. For example, the protein of the present invention can be functionally linked (through chemical coupling, gene fusion, non-covalent linkage or other means) to one or more other molecular groups, such as another protein or polypeptide, detection reagent, pharmaceutical reagent and the like.

In particular, the protein of the present invention can be connected to other functional units. For example, it can be linked to a nuclear localization signal (NLS) sequence to improve the ability of the protein of the present invention to enter the cell nucleus. For example, it can be connected to a targeting moiety to make the protein of the present invention have the targeting. For example, it can be linked to a detectable label to facilitate detection of the protein of the present invention. For example, it can be linked to an epitope tag to facilitate the expression, detection, tracing and/or purification of the protein of the present invention.

### Conjugate

Therefore, in a second aspect, the present invention provides a conjugate comprising the above-mentioned protein and a modified portion.

In certain embodiments, the modified portion is selected from an additional protein or polypeptide, a detectable label, and any combinations thereof.

In certain embodiments, the additional protein or polypeptide is selected from an epitope tag, a reporter gene sequence, a nuclear localization signal (NLS) sequence, a targeting moiety, a transcription activation domain (such as, VP64), a transcription repression domain (for example, KRAB domain or SID domain), a nuclease domain (for example, FokI), a domain having an activity selected from: nucleotide deaminase, methylase activity, demethylase, transcription activation activity, transcription inhibition activity, transcription release factor activity, histone modification activity, nuclease activity, single-stranded RNA cleavage activity, double-stranded RNA cleavage activity, single-stranded DNA cleavage activity, double-stranded DNA cleavage activity, and nucleic acid binding activity; and any combinations thereof.

In certain embodiments, the conjugate of the present invention comprises one or more NLS sequences, such as the NLS of the SV40 virus large T antigen. In certain exemplary embodiments, the NLS sequence is shown in SEQ ID NO: 19. In certain embodiments, the NLS sequence is located at, near, or close to the terminal (such as, N-terminal or C-terminal) of the protein of the present invention. In certain exemplary embodiments, the NLS sequence is located at, near, or close to the C-terminus of the protein of the present invention.

In certain embodiments, the conjugate of the present invention comprises an epitope tag. Such epitope tags are well known to those skilled in the art, examples of which include, but are not limited to, His, V5, FLAG, HA, Myc, VSV-G, Trx, etc., and those skilled in the art know how to select a suitable epitope tag according to the desired purpose (for example, purification, detection or tracing).

In certain embodiments, the conjugate of the present invention comprises a reporter gene sequence. Such reporter genes are well known to those skilled in the art, and examples of which include, but are not limited to GST, HRP, CAT, GFP, HcRed, DsRed, CFP, YFP, BFP and the like.

In certain embodiments, the conjugate of the present invention comprises a domain capable of binding to DNA molecules or intracellular molecules, such as maltose binding protein (MBP), DNA binding domain (DBD) of Lex A, DBD of GAL4, etc.

In certain embodiments, the conjugate of the present invention comprises a detectable label, such as a fluorescent dye, e.g., FITC or DAPI.

In certain embodiments, the protein of the present invention is optionally coupled, conjugated or fused to the modified portion via a linker.

In certain embodiments, the modified portion is directly connected to the N-terminus or C-terminus of the protein of the present invention.

In certain embodiments, the modified portion is connected to the N-terminus or C-terminus of the protein of the present invention through a linker. Such linkers are well known in the art, examples of which include, but are not limited to, a linker containing one or more (for example, 1, 2, 3, 4, or 5) amino acids (such as, Glu or Ser) or amino acid derivatives (such as, Ahx, β-Ala, GABA or Ava) or PEG and the like.

### Fusion Protein

In a third aspect, the present invention provides a fusion protein comprising the protein of the present invention and an additional protein or polypeptide.

In certain embodiments, the additional protein or polypeptide is selected from an epitope tag, a reporter gene sequence, a nuclear localization signal (NLS) sequence, a targeting moiety, a transcription activation domain (such as, VP64), a transcription repression domain (for example, KRAB domain or SID domain), a nuclease domain (for example, FokI), a domain having an activity selected from: a nucleotide deaminase, methylase activity, a demethylase, transcription activation activity, transcription inhibition activity, transcription release factor activity, histone modification activity, nuclease activity, single-stranded RNA cleavage activity, double-stranded RNA cleavage activity, single-stranded DNA cleavage activity, double-stranded DNA cleavage activity, and nucleic acid binding activity; and any combinations thereof.

In certain embodiments, the fusion protein of the present invention comprises one or more NLS sequences, such as the NLS of the SV40 virus large T antigen. In certain embodiments, the NLS sequence is located at, near, or close to the terminal (such as, N-terminal or C-terminal) of the protein of the present invention. In certain exemplary embodiments, the NLS sequence is located at, near, or close to the C-terminus of the protein of the present invention.

In certain embodiments, the fusion protein of the present invention comprises an epitope tag.

In certain embodiments, the fusion protein of the present invention comprises a reporter gene sequence.

In certain embodiments, the fusion protein of the present invention contains a domain capable of binding to DNA molecules or intracellular molecules.

In certain embodiments, the protein of the present invention is optionally fused to the additional protein or polypeptide via a linker.

In certain embodiments, the additional protein or polypeptide is directly linked to the N-terminus or C-terminus of the protein of the present invention.

In certain embodiments, the additional protein or polypeptide is connected to the N-terminus or C-terminus of the protein of the present invention via a linker.

In certain exemplary embodiments, the fusion protein of the present invention has an amino acid sequence as shown in SEQ ID NO: 20.

The protein of the present invention, the conjugate of the present invention, or the fusion protein of the present invention is not limited by the manner in which it is produced. For example, it can be produced by genetic engineering methods (recombinant technology), or can be produced by chemical synthesis methods.

### Direct Repeat

In a fourth aspect, the present invention provides an isolated nucleic acid molecule comprising a sequence selected from the following or consisting of a sequence selected from the following:
(i) a sequence as shown in SEQ ID NO: 7 or 13;
(ii) compared with the sequence as shown in SEQ ID NO: 7 or 13, a sequence having one or more base substitutions, deletions or additions (for example, 1, 2, 3, 4, 5, 6, 7, 8, 9 or 10 base substitutions, deletions or additions);
(iii) a sequence having at least 20%, at least 30%, at least 40%, at least 50%, at least 60%, at least 70%, at least 80%, at least 90%, at least 95% sequence identity with the sequence as shown in SEQ ID NO:7 or 13;
(iv) a sequence that hybridizes to the sequence as described in any one of (i) to (iii) under stringent conditions; or
(v) a complementary sequence of the sequence as described in any one of (i)-(iii);

In addition, the sequence as described in any one of (ii)-(v) substantially retains the biological function of the sequence from which it is derived, and the biological function of the sequence refers to its activity as a direct repeat sequence in the CRISPR-Cas system.

In certain embodiments, the isolated nucleic acid molecule is a direct repeat sequence in the CRISPR-Cas system.

In certain embodiments, the nucleic acid molecule comprises a sequence selected from the following, or consists of a sequence selected from the following:
(a) a nucleotide sequence as shown in SEQ ID NOs: 7 or 13;
(b) a sequence that hybridizes to the sequence as described in (a) under stringent conditions; or
(c) a complementary sequence of the nucleotide sequence as shown in SEQ ID NO: 7 or 13.

In certain embodiments, the isolated nucleic acid molecule is RNA.

### CRISPR/Cas Complex

In a fifth aspect, the present invention provides a complex comprising:
(i) a protein component, which is selected from: the protein, conjugate or fusion protein of the present invention, and any combinations thereof; and
(ii) a nucleic acid component, which comprises the isolated nucleic acid molecule as described in the fourth aspect and a targeting sequence capable of hybridizing to the target sequence from 5′ to 3′ direction,
wherein the protein component and the nucleic acid component combine with each other to form a complex.

In certain embodiments, the targeting sequence is attached to the 3′ end of the nucleic acid molecule.

In certain embodiments, the targeting sequence comprises the complementary sequence of the target sequence.

In certain embodiments, the nucleic acid component is a guide RNA in the CRISPR-Cas system.

In certain embodiments, the nucleic acid molecule is RNA.

In certain embodiments, the complex does not comprise trans-activating crRNA (tracrRNA).

In certain embodiments, the targeting sequence is at least 5, at least 10 in length. In certain embodiments, the targeting sequence is 10-30, or 15-25, or 15-22, or 19-25, or 19-22 nucleotides in length.

In certain embodiments, the isolated nucleic acid molecule is 55-70 nucleotides in length, such as 55-65 nucleotides, such as 60-65 nucleotides, such as 62-65 nucleosides, such as 63-64 nucleotides. In certain embodiments, the isolated nucleic acid molecule is 15-nucleotides in length, such as 15-25 nucleotides, such as 20-25 nucleotides, such as 22-24 nucleotides, such as 23 nucleotides.

In a specific embodiment, the present invention provides a CRISPR-Cas system, which comprises:
a) a guide RNA, which contains a direct repeat sequence and a guide sequence capable of hybridizing to the target sequence from 5′ to 3′ direction, and
b) a Cas12f effector protein;
the guide RNA forms a complex with the Cas12f effector protein;
wherein the Cas 12f protein has a size of 900-1200 amino acids, and there is a RuvC domain near its C-terminus, which is composed of RuvC-I, RuvC-II and RuvC-III motifs;
wherein the Cas12f is located within 500 bp of the CRISPR locus in the bacterial genome;

preferably, the length of the direct repeat sequence is 21nt-36nt, the length of the targeting sequence is 1-80nt, and the last 16 or 17 bases of the direct repeat sequence can form a stem loop, the loop size is 8 or 9 nt, and the stem is composed of five pairs of complementary bases.

### Encoding Nucleic Acid, Vector and Host Cell

In a sixth aspect, the present invention provides an isolated nucleic acid molecule comprising:
(i) a nucleotide sequence encoding the protein or fusion protein of the present invention;
(ii) encoding the isolated nucleic acid molecule as described in the fourth aspect; or
(iii) a nucleotide sequence containing (i) and (ii).

In certain embodiments, the nucleotide sequence described in any one of (i) to (iii) is codon optimized for expression in prokaryotic cells. In certain embodiments, the nucleotide sequence as described in any one of (i) to (iii) is codon optimized for expression in eukaryotic cells.

In a seventh aspect, the present invention also provides a vector comprising the isolated nucleic acid molecule as described in the sixth aspect. The vector of the present invention can be a cloning vector or an expression vector. In certain embodiments, the vector of the present invention is, for example, a plasmid, a cosmid, a bacteriophage, a cosmid and the like. In certain preferred embodiments, the vector is capable of expressing the protein, fusion protein of the present invention, isolated nucleic acid molecule according to the fourth aspect or the complex according to the fifth aspect in a subject (for example, a mammal, such as a human).

In an eighth aspect, the present invention also provides a host cell containing the isolated nucleic acid molecule or vector as described above. Such host cells include, but are not limited to, prokaryotic cells such as E. coli cells, and eukaryotic cells such as yeast cells, insect cells, plant cells and animal cells (such as mammalian cells, e.g., mouse cells, human cells, etc.). The cells of the present invention can also be cell lines, such as 293T cells.

### Composition and Vector Composition

In a ninth aspect, the present invention also provides a composition, which comprises:
(i) a first component, which is selected from: the protein, conjugate, fusion protein of the present invention, nucleotide sequence encoding the protein or fusion protein, and any combinations thereof; and
(ii) a second component, which is a nucleotide sequence containing a guide RNA, or a nucleotide sequence encoding the nucleotide sequence containing a guide RNA;
wherein the guide RNA includes a direct repeat sequence and a targeting sequence from the 5′ to 3′, and the targeting sequence can hybridize with the target sequence;
the targeting RNA can form a complex with the protein, conjugate or fusion protein as described in (i).

In certain embodiments, the direct repeat sequence is an isolated nucleic acid molecule as defined in the fourth aspect.

In certain embodiments, the targeting sequence is connected to the 3′ end of the direct repeat sequence. In certain embodiments, the targeting sequence comprises the complementary sequence of the target sequence.

In certain embodiments, the composition does not include tracrRNA.

In certain embodiments, the composition is non-naturally occurring or modified. In certain embodiments, at least one component of the composition is non-naturally occurring or modified. In certain embodiments, the first component is non-naturally occurring or modified; and/or, the second component is non-naturally occurring or modified.

In certain embodiments, when the target sequence is DNA, the target sequence is located at the 3′ end of the protospacer adjacent motif (PAM), and the PAM has a sequence shown by 5′-TTN, wherein N is selected from A, G, T, and C. In certain embodiments, Nis selected from A, T, and C.

In certain embodiments, when the target sequence is RNA, the target sequence does not have PAM domain restrictions.

In certain embodiments, the target sequence is a DNA or RNA sequence derived from a prokaryotic cell or a eukaryotic cell. In certain embodiments, the target sequence is a non-naturally occurring DNA or RNA sequence.

In certain embodiments, the target sequence is present in the cell. In certain embodiments, the target sequence is present in the cell nucleus or in the cytoplasm (such as, organelles). In certain embodiments, the cell is a eukaryotic cell. In certain embodiments, the cell is a prokaryotic cell.

In certain embodiments, the protein is linked to one or more NLS sequences. In certain embodiments, the conjugate or fusion protein comprises one or more NLS sequences. In certain embodiments, the NLS sequence is linked to the N-terminus or C-terminus of the protein. In certain embodiments, the NLS sequence is fused to the N-terminus or C-terminus of the protein.

In a tenth aspect, the present invention also provides a composition comprising one or more vectors, comprising:
(i) a first nucleic acid, which is a nucleotide sequence encoding a protein or fusion protein of the present invention; optionally, the first nucleic acid is operationally linked to a first regulatory element; and
(ii) a second nucleic acid, which encodes a nucleotide sequence comprising a guide RNA; optionally the second nucleic acid is operationally linked to a second regulatory element;
wherein:
the first nucleic acid and the second nucleic acid are present on the same or different vectors;
the guide RNA includes a direct repeat sequence and a targeting sequence from the 5′ to 3′, and the targeting sequence can hybridize with the target sequence;
the guide RNA can form a complex with the effector protein or fusion protein as described in (i).

In certain embodiments, the direct repeat sequence is an isolated nucleic acid molecule as defined in the fourth aspect.

In certain embodiments, the targeting sequence is connected to the 3′ end of the direct repeat sequence. In certain embodiments, the targeting sequence comprises the complementary sequence of the target sequence.

In certain embodiments, the composition does not include tracrRNA.

In certain embodiments, the composition is non-naturally occurring or modified. In certain embodiments, at least one component of the composition is non-naturally occurring or modified.

In certain embodiments, the first regulatory element is a promoter, such as an inducible promoter.

In certain embodiments, the second regulatory element is a promoter, such as an inducible promoter.

In certain embodiments, when the target sequence is DNA, the target sequence is located at the 3′ end of the protospacer adjacent motif (PAM), and the PAM has a sequence shown by 5′-TTN, wherein N is selected from A, G, T, C. In certain embodiments, N is selected from A, T, and C.

In certain embodiments, when the target sequence is RNA, the target sequence does not have PAM domain restrictions.

In certain embodiments, the target sequence is a DNA or RNA sequence derived from a prokaryotic cell or a eukaryotic cell. In certain embodiments, the target sequence is a non-naturally occurring DNA or RNA sequence.

In certain embodiments, the target sequence is present in the cell. In certain embodiments, the target sequence is present in the cell nucleus or in the cytoplasm (such as, organelles). In certain embodiments, the cell is a eukaryotic cell. In certain embodiments, the cell is a prokaryotic cell.

In certain embodiments, the protein is linked to one or more NLS sequences. In certain embodiments, the conjugate or fusion protein comprises one or more NLS sequences. In certain embodiments, the NLS sequence is linked to the N-terminus or C-terminus of the protein. In certain embodiments, the NLS sequence is fused to the N-terminus or C-terminus of the protein.

In certain embodiments, one type of vector is a plasmid, which refers to a circular double-stranded DNA loop into which additional DNA fragments can be inserted, for example, by standard molecular cloning techniques. Another type of vector is a viral vector, in which virus-derived DNA or RNA sequences are present in the vector used to package the virus (for example, retrovirus, replication-defective retrovirus, adenovirus, replication-defective adenovirus, and adeno-associated vectors also contain polynucleotides carried by the virus used for transfection into a host cell. Certain vectors (for example, bacterial vectors with a bacterial origin of replication and episomal mammalian vectors) are capable of autonomous replication in the host cell into which they are introduced. Other vectors (e.g., non-episomal mammalian vectors) are integrated into the host cell's genome after being introduced into the host cell, and thus replicate with the host genome. Moreover, certain vectors can direct the expression of genes to which they are operationally linked. Such vectors are referred to herein as “expression vectors”. Common expression vectors used in recombinant DNA technology are usually in the form of plasmids.

Recombinant expression vectors may contain the nucleic acid molecule of the present invention in a form suitable for expression of the nucleic acid in a host cell, which means that these recombinant expression vectors contain one or more regulatory elements selected based on the host cell to be used for expression. The regulatory element is operationally linked to the nucleic acid sequence to be expressed.

### Delivery and Delivery Composition

The protein, conjugate, fusion protein of the present invention, the isolated nucleic acid molecule as described in the fourth aspect, the complex of the present invention, the isolated nucleic acid molecule as described in the sixth aspect, the vector as described in the seventh aspect, the composition as described in the ninth and tenth aspects can be delivered by any method known in the art. Such methods include, but are not limited to, electroporation, lipofection, nuclear transfection, microinjection, sonoporation, gene gun, calcium phosphate-mediated transfection, cationic transfection, liposome transfection, dendritic transfection, heat shock transfection, nuclear transfection, magnetic transfection, lipofection, puncture transfection, optical transfection, reagent-enhanced nucleic acid uptake, and delivery via liposome, immunoliposome, viral particle, artificial virosome etc.

Therefore, in another aspect, the present invention provides a delivery composition comprising a delivery vehicle and one or more selected from the following: the protein, conjugate, fusion protein of the present invention, the isolated nucleic acid molecule according to the fourth aspect, the complex of the present invention, the isolated nucleic acid molecule according to the sixth aspect, the vector according to the seventh aspect, the composition according to the ninth and tenth aspects.

In certain embodiments, the delivery vehicle is a particle.

In certain embodiments, the delivery vehicle is selected from a lipid particle, sugar particle, metal particle, protein particle, liposome, exosome, microvesicle, gene gun, or viral vector (e.g., replication defective retrovirus, lentivirus, adenovirus or adeno-associated virus).

### Kit

In another aspect, the present invention provides a kit comprising one or more of the components as described above. In certain embodiments, the kit includes one or more components selected from the following: the protein, conjugate, fusion protein of the present invention, the isolated nucleic acid molecule as described in the fourth aspect, the complex of the present invention, the isolated nucleic acid molecule as described in the sixth aspect, the vector as described in the seventh aspect, and the composition as described in the ninth and tenth aspects.

In certain embodiments, the kit of the present invention comprises the composition as described in the ninth aspect. In certain embodiments, the kit further includes instructions for using the composition.

In certain embodiments, the kit of the present invention comprises a composition as described in the tenth aspect. In certain embodiments, the kit further includes instructions for using the composition.

In certain embodiments, the component contained in the kit of the present invention may be provided in any suitable container.

In certain embodiments, the kit further includes one or more buffers. The buffer can be any buffer, including but not limited to sodium carbonate buffer, sodium bicarbonate buffer, borate buffer, Tris buffer, MOPS buffer, HEPES buffer, and combinations thereof. In certain embodiments, the buffer is alkaline. In certain embodiments, the buffer has a pH of from about 7 to about 10.

In certain embodiments, the kit further includes one or more oligonucleotides corresponding to a targeting sequence for insertion into the vector so as to link the targeting sequence and regulatory element operationally. In certain embodiments, the kit includes a homologous recombination template polynucleotide.

### Method and Use

In another aspect, the present invention provides a method for modifying a target gene, which comprises: contacting the complex according to the fifth aspect, the composition according to the ninth aspect, or the composition according to the tenth aspect with the target gene, or delivering that to a cell containing the target gene; the target sequence is present in the target gene.

In certain embodiments, the target gene is present in the cell. In certain embodiments, the cell is a prokaryotic cell. In certain embodiments, the cell is a eukaryotic cell. In certain embodiments, the cell is a mammalian cell. In certain embodiments, the cell is a human cell. In certain embodiments, the cell is selected from a non-human primate, bovine, pig, or rodent cell. In certain embodiments, the cell is a non-mammalian eukaryotic cell, such as poultry or fish and the like. In certain embodiments, the cell is a plant cell, such as a cell possessed by a cultivated plant (such as cassava, corn, sorghum, wheat, or rice), algae, tree, or vegetable.

In certain embodiments, the target gene is present in a nucleic acid molecule (e.g., a plasmid) in vitro. In certain embodiments, the target gene is present in a plasmid.

In certain embodiments, the modification refers to a break in the target sequence, such as a double-strand break in DNA or a single-strand break in RNA.

In certain embodiments, the break results in decreased transcription of the target gene.

In certain embodiments, the method further comprises: contacting the editing template with the target gene, or delivering it to the cell containing the target gene. In such embodiments, the method repairs the broken target gene by homologous recombination with an exogenous template polynucleotide, wherein the repair results in a mutation including the insertion, deletion, or substitution of one or more nucleotides of the target gene. In certain embodiments, the mutation results in one or more amino acid changes in the protein expressed from the gene containing the target sequence.

Therefore, in certain embodiments, the modification further includes inserting an editing template (for example, an exogenous nucleic acid) into the break.

In certain embodiments, the protein, conjugate, fusion protein, isolated nucleic acid molecule, complex, vector or composition is contained in a delivery vehicle.

In certain embodiments, the delivery vehicle is selected from a lipid particle, sugar particle, metal particle, protein particle, liposome, exosome, viral vector (such as replication-defective retrovirus, lentivirus, adenovirus or adeno-associated virus).

In certain embodiments, the method is used to change one or more target sequences in a target gene or a nucleic acid molecule encoding a target gene product to modify a cell, cell line, or organism.

In another aspect, the present invention provides a method for altering the expression of a gene product, which comprises: contacting the complex according to the fifth aspect, the composition according to the ninth aspect or the composition according to the tenth aspect with a nucleic acid molecule encoding the gene product, or delivering that to a cell containing the nucleic acid molecule in which the target sequence is present.

In certain embodiments, the nucleic acid molecule is present in a cell. In certain embodiments, the cell is a prokaryotic cell. In certain embodiments, the cell is a eukaryotic cell. In certain embodiments, the cell is a mammalian cell. In certain embodiments, the cell is a human cell. In certain embodiments, the cell is selected from a non-human primate, bovine, pig, or rodent cell. In certain embodiments, the cell is a non-mammalian eukaryotic cell, such as poultry or fish and the like. In certain embodiments, the cell is a plant cell, such as a cell possessed by a cultivated plant (such as cassava, corn, sorghum, wheat, or rice), algae, tree, or vegetable.

In certain embodiments, the nucleic acid molecule is present in a nucleic acid molecule (e.g., a plasmid) in vitro. In certain embodiments, the nucleic acid molecule is present in a plasmid.

In certain embodiments, the expression of the gene product is altered (e.g., enhanced or decreased). In certain embodiments, the expression of the gene product is enhanced. In certain embodiments, the expression of the gene product is reduced.

In certain embodiments, the gene product is a protein.

In certain embodiments, the protein, conjugate, fusion protein, isolated nucleic acid molecule, complex, vector or composition is contained in a delivery vehicle.

In certain embodiments, the delivery vehicle is selected from a lipid particle, sugar particle, metal particle, protein particle, liposome, exosome, viral vector (such as replication-defective retrovirus, lentivirus, adenovirus or adeno-associated virus).

In certain embodiments, the method is used to change one or more target sequences in a target gene or a nucleic acid molecule encoding a target gene product to modify a cell, cell line, or organism.

In another aspect, the present invention relates to a use of the protein according to the first aspect, the conjugate according to the second aspect, the fusion protein according to the third aspect, the isolated nucleic acid molecule according to the fourth aspect, the complex according to the fifth aspect, the isolated nucleic acid molecule according to the sixth aspect, the vector according to the seventh aspect, the composition according to the ninth aspect, the composition according to the tenth aspect of the present invention, the kit or delivery composition of the present invention for the nucleic acid editing.

In certain embodiments, the nucleic acid editing includes gene or genome editing, such as modifying genes, knocking out genes, altering the expression of gene products, repairing mutations, and/or inserting polynucleotides.

In another aspect, the present invention relates to a use of the protein according to the first aspect, the conjugate according to the second aspect, the fusion protein according to the third aspect, the isolated nucleic acid molecule according to the fourth aspect, the complex according to the fifth aspect, the isolated nucleic acid molecule according to the sixth aspect, the vector according to the seventh aspect, the composition according to the ninth aspect, the composition according to the tenth aspect of the present invention, the kit or delivery composition of the present invention in the preparation of a formulation, which is used for:
(i) the isolated gene or genome editing;
(ii) the detection of an isolated single-stranded DNA;
(iii) editing the target sequence in the target locus to modify a organism or non-human organism;
(iv) treating the disease caused by defects in the target sequence in the target locus.

### Cells and Cell Progeny

In some cases, the modifications introduced into the cell by the method of the present invention can cause the cell and its progeny to be altered to improve the production of its biological products (such as antibodies, starch, ethanol, or other desired cell output). In some cases, the modifications introduced into the cell by the methods of the present invention can cause the cell and its progeny to include changes that alter the biological product produced.

Therefore, in another aspect, the present invention also relates to a cell or its progeny obtained by the method as described above, wherein the cell contains a modification that is not present in its wild type.

The present invention also relates to the cell product of the cell or its progeny as described above.

The present invention also relates to an in vitro, isolated or in vivo cell or cell line or their progeny, the cell or cell line or their progeny comprises: the protein according to the first aspect, the conjugate according to the second aspect, the fusion protein according to the third aspect, the isolated nucleic acid molecule according to the fourth aspect, the complex according to the fifth aspect, the isolated nucleic acid molecule according to the sixth aspect, the vector according to the seventh aspect, the composition according to the ninth aspect, the composition according to the tenth aspect of the present invention, the kit or delivery composition of the present invention.

In certain embodiments, the cell is a prokaryotic cell.

In certain embodiments, the cell is an eukaryotic cell. In certain embodiments, the cell is a mammalian cell. In certain embodiments, the cell is a human cell. In certain embodiments, the cell is a non-human mammalian cell, such as a cell of a non-human primate, cow, sheep, pig, dog, monkey, rabbit, rodent (e.g., rat or mouse). In certain embodiments, the cell is a non-mammalian eukaryotic cell, such as a poultry bird (e.g., chicken), fish, or crustacean (e.g., clam, shrimp) cell. In certain embodiments, the cell is a plant cell, such as a cell possessed by a monocotyledon or dicotyledon or a cell possessed by a cultivated plant or a food crop such as cassava, corn, sorghum, soybean, wheat, oats or rice, such as algae, trees or production plants, fruits or vegetables (for example, trees such as citrus trees, nut trees; nightshades, cotton, tobacco, tomatoes, grapes, coffee, cocoa, etc.).

In certain embodiments, the cell is a stem cell or stem cell line.

### DEFINITION OF TERMS

In the present invention, unless otherwise specified, the scientific and technical terms used herein have the meanings commonly understood by those skilled in the art. In addition, the molecular genetics, nucleic acid chemistry, chemistry, molecular biology, biochemistry, cell culture, microbiology, cell biology, genomics and recombinant DNA and other procedures used in this article are all routine procedures widely used in the corresponding fields. At the same time, in order to better understand the present invention, definitions and explanations of related terms are provided below.

In the present invention, the expression “Cas12f” refers to a Cas effector protein discovered and identified for the first time by the present inventors, which has an amino acid sequence selected from the following:
(i) a sequence as shown in any one of SEQ ID NOs: 1, 2, 3;
(ii) compared with the sequence as shown in any one of SEQ ID NOs: 1, 2, 3, a sequence having one or more amino acid substitutions, deletions or additions (for example, 1, 2, 3, 4, 5, 6, 7, 8, 9 or 10 amino acid substitutions, deletions or additions); or
(iii) a sequence having at least 20%, at least 30%, at least 40%, at least 50%, at least 60%, at least 70%, at least 80%, at least 80%, at least 85%, at least 90%, at least 91%, at least 92%, at least 93%, at least 94%, at least 95%, at least 96%, at least 97%, at least 98%, or at least 99% sequence identity with the sequence as shown in any one of SEQ ID NOs: 1, 2, 3.

The Cas12f of the present invention is an endonuclease that binds to and cleaves a specific site of a target sequence under the guidance of a guide RNA, and has DNA and RNA endonuclease activities at the same time.

As used herein, the terms “Clustered Regularly Interspaced Short Palindromic Repeats (CRISPR)-CRISPR-associated (Cas) (CRISPR-Cas) system” or “CRISPR system” are used interchangeably and have the meaning commonly understood by those skilled in the art, it usually contains transcription products or other elements related to the expression of CRISPR-associated (“Cas”) genes, or transcription products or other elements capable of directing the activity of the Cas gene. Such transcription products or other elements may include sequences encoding Cas effector proteins and guide RNAs including CRISPR RNA (crRNA), as well as trans-activating crRNA (tracrRNA) sequences contained in the CRISPR-Cas9 system, or other sequences or transcription products from the CRISPR locus. In the Cas12f-based CRISPR system of the present invention, the tracrRNA sequence is not required.

As used herein, the terms “Cas effector protein” and “Cas effector enzyme” are used interchangeably and refer to any protein present in the CRISPR-Cas system that is greater than 900 amino acids in length. In some cases, this type of protein refers to a protein identified from the Cas locus.

As used herein, the terms “guide RNA” and “mature crRNA” can be used interchangeably and have meanings commonly understood by those skilled in the art. Generally speaking, a guide RNA can contain a direct repeat and a guide sequence, or it essentially consists of or consists of a direct repeat sequence and a targeting sequence (also called a spacer in the context of an endogenous CRISPR system). In some cases, the targeting sequence is any polynucleotide sequence that has sufficient complementarity with the target sequence to hybridize to the target sequence and guide the specific binding of the CRISPR/Cas complex to the target sequence. In certain embodiments, when optimally aligned, the degree of complementarity between the targeting sequence and its corresponding target sequence is at least 50%, at least 60%, at least 70%, at least 80%, at least 90%, at least 95%, or at least 99%. Determining the best alignment is within the ability of a person of ordinary skill in the art. For example, there are published and commercially available alignment algorithms and programs, such as but not limited to ClustalW, Smith-Waterman algorithm in matlab, Bowtie, Geneious, Biopython, and SeqMan.

In some cases, the targeting sequence is at least 5, at least 10, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, or at least 50 nucleotides in length. In some cases, the targeting sequence is no more than 50, 45, 40, 35, 30, 25, 24, 23, 22, 21, 20, 15, 10 or fewer nucleotides in length. In certain embodiments, the targeting sequence is 10-30, or 15-25, or 15-22, or 19-25, or 19-22 nucleotides in length.

In some cases, the direct repeat sequence is at least 10, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 56, at least 57, at least 58, at least 59, at least 60, at least 61, at least 62, at least 63, at least 64, at least 65, or at least 70 nucleotides in length. In some cases, the direct repeat sequence is no more than 70, 65, 64, 63, 62, 61, 60, 59, 58, 57, 56, 55, 50, 45, 40, 35, 30, 29, 28, 27, 26, 25, 24, 23, 22, 21, 20, 15, 10 or fewer nucleotides in length. In certain embodiments, the direct repeat sequence is 55-70 nucleotides in length, such as 55-65 nucleotides, such as 60-65 nucleotides, such as 62-65 nucleotides, such as 63-64 nucleotides. In certain embodiments, the direct repeat sequence is 15-30 nucleotides in length, such as 15-25 nucleotides, such as 20-25 nucleotides, such as 22-24 nucleosides, such as 23 nucleotides.

As used herein, the term “CRISPR/Cas complex” refers to a ribonucleoprotein complex formed by the combination of guide RNA or mature crRNA and Cas protein, which contains a targeting sequence that hybridizes to the target sequence and binds to the Cas protein. The ribonucleoprotein complex can recognize and cleave polynucleotides that can hybridize with the guide RNA or mature crRNA.

Therefore, in the case of forming a CRISPR/Cas complex, the “target sequence” refers to a polynucleotide that is targeted by a targeting sequence designed to have targeting, for example, a sequence that is complementary to the targeting sequence, wherein the hybridization between the target sequence and the targeting sequence will promote the formation of the CRISPR/Cas complex. Complete complementarity is not necessary, as long as there is sufficient complementarity to cause hybridization and promote the formation of a CRISPR/Cas complex. The target sequence can comprise any polynucleotide, such as DNA or RNA. In some cases, the target sequence is located in the nucleus or cytoplasm of the cell. In some cases, the target sequence may be located in an organelle of an eukaryotic cell such as mitochondria or chloroplast. The sequence or template that can be used to be recombined into the target locus containing the target sequence is referred to as “editing template” or “editing polynucleotide” or “editing sequence”. In certain embodiments, the editing template is an exogenous nucleic acid. In certain embodiments, the recombination is a homologous recombination.

In the present invention, the expression “target sequence” or “target polynucleotide” can be any endogenous or exogenous polynucleotide for a cell (for example, a eukaryotic cell). For example, the target polynucleotide may be a polynucleotide present in the nucleus of a eukaryotic cell. The target polynucleotide may be a sequence encoding a gene product (e.g., protein) or a non-coding sequence (e.g., regulatory polynucleotide or useless DNA). In some cases, it is believed that the target sequence should be related to the protospacer adjacent motif (PAM). The exact sequence and length requirements for PAM vary depending on the Cas effector enzyme used, but PAM is typically a 2-5 base pair sequence adjacent to the protospacer (i.e., the target sequence). Those skilled in the art are able to identify the PAM sequence to be used with a given Cas effector protein.

In some cases, the target sequence or target polynucleotide may include multiple disease-related genes and polynucleotides and signal transduction biochemical pathway-related genes and polynucleotides. Non-limiting examples of such target sequences or target polynucleotides include those listed in U.S. Provisional Patent Applications 61/736,527 and 61/748,427 filed on Dec. 12, 2012 and Jan. 2, 2013, respectively, and the international application PCT/US2013/074667 filed on Dec. 12, 2013, which are all incorporated herein by reference.

In some cases, examples of a target sequence or a target polynucleotide includes a sequence related to signaling biochemical pathways, such as a signaling biochemical pathway related gene or polynucleotide. Examples of a target polynucleotide includes a disease-related gene or polynucleotide. The “disease-related” gene or polynucleotide refers to any gene or polynucleotide that produces transcription or translation products at abnormal levels or in abnormal forms in cells derived from tissues affected by the disease, compared with non-disease control tissues or cells. In the case where the altered expression is related to the appearance and/or progression of the disease, it may be a gene expressed at an abnormally high level; or, it may be a gene expressed at an abnormally low level. The disease-related gene also refers to genes that have one or more mutations or genetic variations that are directly responsible for or genetic linkage disequilibrium with one or more genes responsible for the etiology of the disease. The transcribed or translated product can be known or unknown, and can be at normal or abnormal levels.

As used herein, the term “wild-type” has the meaning commonly understood by those skilled in the art, which means a typical form of organisms, strains, genes, or features that distinguish it from mutants or variant forms when it exists in nature, it can be isolated from natural sources and has not been deliberately modified.

As used herein, the terms “non-naturally occurring” or “engineered” can be used interchangeably and refer to artificial involvement. When these terms are used to describe a nucleic acid molecule or polypeptide, it means that the nucleic acid molecule or polypeptide is at least substantially free from at least another component that they bind to in nature or as found in nature.

As used herein, the term “orthologue (ortholog)” has the meaning commonly understood by those skilled in the art. As a further guidance, the “orthologue” of the protein as described herein refers to proteins belonging to different species, which perform the same or similar functions as the proteins that act as their orthologs.

As used herein, the term “identity” is used to refer to the matching of sequences between two polypeptides or between two nucleic acids. When a certain position in the two sequences to be compared is occupied by the same base or amino acid monomer subunit (for example, a certain position in each of the two DNA molecules is occupied by adenine, or a certain position in each of the two peptides is occupied by lysine), then the molecules are identical at that position. The “percent identity” between two sequences is a function of the number of matching positions shared by the two sequences divided by the number of positions to be compared×100. For example, if 6 out of 10 positions in two sequences match, then the two sequences have 60% identity. For example, the DNA sequences CTGACT and CAGGTT share 50% identity (3 out of 6 total positions match). Generally, the comparison is made when two sequences are aligned to produce maximum identity. Such alignment can be achieved by using, for example, the method of Needleman et al. (1970) J. Mol. Biol. 48:443-453, which can be conveniently performed by a computer program such as the Align program (DNAstar, Inc.). It is also possible to use the algorithm of E. Meyers and W. Miller (Comput. Appl Biosci., 4:11-17 (1988)) integrated into the ALIGN program (version 2.0), using the PAM120 weight residue table, a gap length penalty of 12, and a gap penalty of 4 to determine the percent identity between two amino acid sequences. In addition, the Needleman and Wunsch (J Mol Biol. 48:444-453 (1970)) algorithm in the GAP program integrated into the GCG software package (available on www.gcg.com) can be used, the Blossum 62 matrix or PAM250 matrix and gap weights of 16, 14, 12, 10, 8, 6, or 4 and length weights of 1, 2, 3, 4, 5 or 6 to determine the percent identity between two amino acid sequences.

As used herein, the term “vector” refers to a nucleic acid delivery vehicle into which a polynucleotide can be inserted. When the vector can express the protein encoded by the inserted polynucleotide, the vector is called an expression vector. The vector can be introduced into the host cell through transformation, transduction or transfection, so that the genetic material elements which it carries can be expressed in the host cell. Vector is well-known to those skilled in the art, including but not limited to: a plasmid; phagemid; cosmid; artificial chromosome, such as yeast artificial chromosome (YAC), bacterial artificial chromosome (BAC) or P1 derived artificial chromosome (PAC); bacteriophage such as a lambda bacteriophage or M13 bacteriophage and animal virus. An animal virus that can be used as a vector includes, but is not limited to, a retrovirus (including a lentivirus), adenovirus, adeno-associated virus, herpes virus (such as herpes simplex virus), poxvirus, baculovirus, papilloma virus, and papovaviruses (such as SV40). A vector can contain a variety of elements that control expression, including but not limited to a promoter sequence, transcription initiation sequence, enhancer sequence, selection element, and reporter gene. In addition, the vector may also contain an origin of replication.

As used herein, the term “host cell” refers to a cell that can be used to introduce a vector, which includes, but is not limited to, a prokaryotic cell such as Escherichia coli or Bacillus subtilis and the like, a fungal cell such as a yeast cell or Aspergillus, etc., an insect cell such as a S2 Drosophila cell or Sf9, etc., or an animal cell such as a fibroblast, CHO cell, COS cell, NSO cell, HeLa cell, BHK cell, HEK 293 cell or human cell, etc.

Those skilled in the art will understand that the design of the expression vector may depend on factors such as the selection of the host cell to be transformed, the desired expression level, and the like. A vector can be introduced into a host cell to thereby produce transcripts, proteins, or peptides, including proteins, fusion proteins, isolated nucleic acid molecules, etc. as described herein (for example, CRISPR transcripts, such as nucleic acid transcripts, proteins, or enzymes).

As used herein, the term “regulatory element” is intended to include a promoter, enhancer, internal ribosome entry site (IRES), and other expression control elements (e.g., transcription termination signals, such as polyadenylation signals and Poly U sequence), for a detailed description, please refer to Goeddel, “GENE EXPRESSION TECHNOLOGY: METHOD IN ENZYMOLOGY” 185, Academic Press, San Diego, California (1990). In some cases, the regulatory element includes those that direct the constitutive expression of a nucleotide sequence in many types of host cells and those that direct the expression of the nucleotide sequence only in certain host cells (for example, tissue-specific regulatory sequence). A tissue-specific promoter may mainly direct expression in desired tissues of interest, such as muscles, neurons, bone, skin, blood, specific organs (such as liver, pancreas), or specific cell types (such as lymphocytes). In some cases, the regulatory element may also direct expression in a time-dependent manner (such as in a cell cycle-dependent or developmental stage-dependent manner), which may be or may not be tissue or cell type specific. In some cases, the term “regulatory element” encompasses an enhancer element, such as WPRE; a CMV enhancer; R-U5′ fragment in the LTR of HTLV-I ((Mol. Cell. Biol., Volume 8 (1), Pages 466-472, 1988); SV40 enhancer; and the intron sequence between exons 2 and 3 of rabbit β-globin (Proc. Natl. Acad. Sci. USA., Vol. 78 (3), pp. 1527-31, 1981).

As used herein, the term “promoter” has the meaning well known to those skilled in the art, which refers to a non-coding nucleotide sequence located upstream of a gene and capable of promoting downstream gene expression. A constitutive promoter is such a nucleotide sequence: when it is operationally linked to a polynucleotide encoding or defining a gene product, it leads to the production of a gene product in the cell under most or all physiological conditions of the cell. An inducible promoter is such a nucleotide sequence that, when operationally linked to a polynucleotide encoding or defining a gene product, basically only when an inducer corresponding to the promoter is present in the cell, it leads to the gene product to be produced in the cell. A tissue-specific promoter is such a nucleotide sequence that, when operationally linked to a polynucleotide encoding or defining a gene product, basically only when the cell is a cell of the tissue type corresponding to the promoter, it leads to the production of gene products in the cell.

As used herein, the term “operationally linked” is intended to mean that the nucleotide sequence of interest is linked to the one or more regulatory elements in a manner that allows the expression of the nucleotide sequence (e.g., in an in vitro transcription/translation system or when the vector is introduced into the host cell, it is in the host cell).

As used herein, the term “complementarity” refers to the ability of a nucleic acid to form one or more hydrogen bonds with another nucleic acid sequence by means of traditional Watson-Crick or other non-traditional types. The percentage of complementarity represents the percentage of residues in a nucleic acid molecule that can form hydrogen bonds (e.g., Watson-Crick base pairing) with a second nucleic acid sequence (e.g., 5, 6, 7, 8, 9, 10 out of 10 are 50%, 60%, 70%, 80%, 90%, and 100% complementary). “Completely complementary” means that all consecutive residues of a nucleic acid sequence form hydrogen bonds with the same number of consecutive residues in a second nucleic acid sequence. As used herein, “substantially complementary” means that there are at least 60%, 65%, 70%, 75%, 80%, 85%, 90%, 95%, 97%, 98%, 99%, or 100% degree of complementarity in a region of 8, 9, 10, 11, 12, 13, 14, 15, 16, 17, 18, 19, 20, 21, 22, 23, 24, 25, 30, 35, 40, 45, 50 or more nucleotides, or refers to two nucleic acids that hybridize under stringent conditions.

As used herein, “stringent conditions” for hybridization refer to conditions under which a nucleic acid having complementarity with a target sequence mainly hybridizes to the target sequence and substantially does not hybridize to a non-target sequence. Stringent conditions are usually sequence-dependent and vary depending on many factors. Generally speaking, the longer the sequence, the higher the temperature at which the sequence specifically hybridizes to its target sequence. Non-limiting examples of stringent conditions are described in “Laboratory Techniques In Biochemistry And Molecular Biology-Hybridization With Nucleic Acid Probes” by Tijssen (1993), Part I, Chapter 2, “Overview of principles of hybridization and the strategy of nucleic acid probe assay”, Elsevier, New York.

As used herein, the term “hybridization” refers to a reaction in which one or more polynucleotides react to form a complex that is stabilized by hydrogen bonding of bases between these nucleotide residues. Hydrogen bonding can occur by means of Watson-Crick base pairing, Hoogstein binding, or in any other sequence-specific manner. The complex may comprise two strands forming a duplex, three or more strands forming a multi-strand complex, a single self-hybridizing strand, or any combination of these. The hybridization reaction can constitute a step in a broader process (such as the beginning of PCR, or the cleavage of polynucleotides by an enzyme). A sequence that can hybridize to a given sequence is called the “complement” of the given sequence.

As used herein, the term “expression” refers to the process by which the DNA template is transcribed into polynucleotides (such as mRNA or other RNA transcripts) and/or the process by which the transcribed mRNA is subsequently translated into peptides, polypeptides or proteins. The transcript and the encoded polypeptide can be collectively referred to as a “gene product”. If the polynucleotide is derived from a genomic DNA, the expression can include splicing of mRNA in eukaryotic cells.

As used herein, the term “linker” refers to a linear polypeptide formed by multiple amino acid residues connected by peptide bonds. The linker of the present invention may be an artificially synthesized amino acid sequence, or a naturally-occurring polypeptide sequence, such as a polypeptide having the function of a hinge region. Such linker polypeptides are well known in the art (see, for example, Holliger, P. et al. (1993) Proc. Natl. Acad. Sci. USA 90:6444-6448; Poljak, R J et al. (1994) Structure 2:1121-1123).

As used herein, the term “treatment” refers to treating or curing a disorder, delaying the onset of symptoms of the disorder, and/or delaying the development of the disorder.

As used herein, the term “subject” includes, but is not limited to, various animals, such as mammals, e.g., bovines, equines, caprids, swines, canines, felines, leporidae animals, rodents (for example, mice or rats), non-human primates (for example, macaques or cynomolgus), or humans. In certain embodiments, the subject (e.g., human) has a disorder (e.g., a disorder caused by a disease-related gene defect).

### The Beneficial Effects of the Present Invention

Compared with the prior art, the Cas protein and system of the present invention have significant advantages. For example, the PAM domain of the Cas effector protein of the present invention is a strict 5′-TTN structure, and nearly 100% of the second and third bases in front of the target sequence are T, and the other positions can be arbitrary sequences. It has a more rigorous PAM recognition method than the most rigorous PAM recognition C2c1 that has been reported so far, which significantly reduces off-target effects. For example, the Cas effector protein of the present invention can perform DNA cleavage in eukaryotes, and its molecular size is about 200-300 amino acids smaller than Cpf1 and Cas9 proteins, so that the transfection efficiency is significantly better than Cpf1 and Cas9.

The embodiments of the present invention will be described in detail below in conjunction with the accompanying drawings and examples. However, those skilled in the art will understand that the following drawings and examples are only used to illustrate the present invention, but not to limit the scope of the present invention. According to the accompanying drawings and the following detailed description of the preferred embodiments, various objects and advantageous aspects of the present invention will become apparent to those skilled in the art.

### BRIEF DESCRIPTION OF THE DRAWINGS

FIG. 1 is the result of the crRNA structure analysis of Cas12f.4, Cas12f.5 and Cas12f.6 in Example 2, showing the secondary structure of the Repeat sequence.

FIG. 2 shows the analysis result of the PAM domain in Example 3.

FIGS. 3A-FIG. 3C are the results of the detection of the cleavage activity of Cas12f.4 in a human cell line in Example 4.

FIGS. 4A-FIG. 4C are the results of the detection of the cleavage activity of Cas12f.4 in a maize protoplast cell in Example 5.

### SEQUENCE INFORMATION

Information on partial sequences involved in the present invention is provided in Table 1 below.

TABLE 1
Description of the sequence

| SEQ ID NO: | Description |
|---|---|
| 1 | an amino acid sequence of Cas12f.4 |
| 2 | an amino acid sequence of Cas12f.5 |
| 3 | an amino acid sequence of Cas12f.6 |
| 4 | a coding nucleic acid sequence of Cas12f.4 |
| 5 | a coding nucleic acid sequence of Cas12f.5 |
| 6 | a coding nucleic acid sequence of Cas12f.6 |
| 7 | Cas12f.4/prototype direct repeat |
| 8 | Cas12f.5/prototype direct repeat |
| 9 | Cas12f.6/prototype direct repeat |
| 10 | Cas12f.4/a coding nucleic acid sequence of prototype |
| | direct repeat |
| 11 | Cas12f.5/a coding nucleic acid sequence of prototype |
| | direct repeat |
| 12 | Cas12f.6/a coding nucleic acid sequence of prototype |
| | direct repeat |
| 13 | Cas12f.4/mature direct repeat |
| 14 | Cas12f.5/mature direct repeat |
| 15 | Cas12f.6/mature direct repeat |
| 16 | Cas12f.4/a coding nucleic acid sequence of mature direct |
| | repeat |
| 17 | Cas12f.5/a coding nucleic acid sequence of mature direct |
| | repeat |
| 18 | Cas12f.6/a coding nucleic acid sequence of mature direct |
| | repeat |
| 19 | NLS sequence |
| 20 | an amino acid sequence of Cas12f.4-NLS fusion protein |
| 21 | an amino acid sequence of Cas12f.5-NLS fusion protein |
| 22 | an amino acid sequence of Cas12f.6-NLS fusion protein |
| 23 | a plasmid expressing Cas12f.4 system |
| 24 | PAM library sequence |
| 25 | guide RNA-VEGFA of Cas12f.4 system |
| 26 | guide RNA-VEGFA of Cas12f.5 system |
| 27 | guide RNA-VEGFA of Cas12f.6 system |
| 28 | guide RNA-PDI1 of Cas12f.4 system |
| 29 | guide RNA-SBE2.2 of Cas12f.4 system |

### DETAILED DESCRIPTION OF THE EMBODIMENTS

The invention will now be described with reference to the following examples which are intended to illustrate the present invention rather than limit the present invention.

Unless otherwise specified, the experiments and methods described in the examples are basically performed according to conventional methods well known in the art and described in various references. For example, conventional techniques such as immunology, biochemistry, chemistry, molecular biology, microbiology, cell biology, genomics, and recombinant DNA used in the present invention can be found in Sambrook, Fritsch and Maniatis, “MOLECULAR CLONING: A LABORATORY MANUAL”, 2nd edition (1989); “CURRENT PROTOCOLS IN MOLECULAR BIOLOGY” (edited by F. M. Ausubel et al., (1987)); “METHODS IN ENZYMOLOGY” series (Academic Publishing Company): “PCR 2: A PRACTICAL APPROACH” (edited by M. J. MacPherson, BD Hames and G. R. Taylor (1995)), “ANTIBODIES, A LABORATORY MANUAL”, edited by Harlow and Lane (1988), and “ANIMAL CELL CULTURE” (edited by R. I. Freshney (1987)).

In addition, if the specific conditions are not specified in the examples, it shall be carried out in accordance with the conventional conditions or the conditions recommended by the manufacturer. The reagents or instruments used without the manufacturer's indication are all conventional products that can be purchased commercially. Those skilled in the art know that the embodiments describe the present invention by way of example, and are not intended to limit the scope of protection claimed by the present invention. All publications and other references mentioned in this article are incorporated into this article by reference in their entirety.

The sources of some reagents involved in the following examples are as follows:
LB liquid medium: 10 g Tryptone, 5 g Yeast Extract, 10 g NaCl, diluted to 1 L, and sterilized. If antibiotics are needed, they are added at a final concentration of 50 μg/ml after cooling the medium.

Chloroform/isoamyl alcohol: adding 240 ml of chloroform to 10 ml of isoamyl alcohol and mixing them well.

RNP buffer: 100 mM sodium chloride, 50 mM Tris-HCl, 10 mM MgCl₂, 100 μg/ml BSA, pH 7.9.

The prokaryotic expression vectors pACYC-Duet-1 and pUC19 are purchased from Beijing Quanshijin Biotechnology Co., Ltd.

E. coli competence EC100 is purchased from Epicentre company.

### Example 1. Acquisition of Cas12f Gene and Cas12f Guide RNA

1. CRISPR and gene annotation: Using Prodigal to perform gene annotation on the microbial genome and metagenomic data of NCBI and JGI databases to obtain all proteins and at the same time, using Piler-CR to annotate CRISPR locus. All parameters are the default parameters.
2. Protein filtering: Eliminating redundancy of annotated proteins by sequence identity, removing proteins with exactly identical sequence, and at the same time classifying proteins longer than 800 amino acids into macromolecular proteins. Since all the effector proteins of the second type of CRISPR/Cas system discovered so far are more than 900 amino acids in length, in order to reduce the computational complexity, when we explore CRISPR effector proteins, we only consider macromolecular proteins.
3. Obtaining CRISPR-associated macromolecular proteins: extending each CRISPR locus by 10 Kb upstream and downstream, and identifying non-redundant macromolecular proteins in the adjacent interval of CRISPR.
4. Clustering of CRISPR-associated macromolecular proteins: using BLASTP to perform internal pairwise comparisons of non-redundant macromolecular CRISPR-associated proteins, and output the comparison result of Evalue<1E-10. Using MCL to perform cluster analysis on the output result of BLASTP, CRISPR-associated protein family.
5. Identification of CRISPR-enriched macromolecular protein family: using BLASTP to compare the proteins of the CRISPR-associated protein family to the non-redundant macromolecular protein database that removes the CRISPR-associated proteins and output the comparison result of Evalue<1E-10. If the homologous protein found in a non-CRISPR-related protein database is less than 100%, it means that the proteins of this family are enriched in the CRISPR region. In this way, we identify the CRISPR-enriched macromolecular protein family.
6. Annotation of protein functions and domains: using the Pfam database, NR database and Cas protein collected from NCBI to annotate the CRISPR-enriched macromolecular protein family to obtain a new CRISPR/Cas protein family. Using Mafft to perform multiple sequence alignments for each CRISPR/Cas family protein, and then using JPred and HHpred to perform conserved domain analysis to identify protein families containing RuvC domains.

On this basis, the present inventors have obtained a new Cas effector protein, namely Cas12f, named Cas12f.4 (SEQ ID NO: 1), Cas12f.5 (SEQ ID NO: 2) and Cas12f.6 (SEQ ID NO: 3), respectively with its three active homologue sequences, the coding DNA of the three homologues are shown in SEQ ID NOs: 4, 5, and 6, respectively. The prototype direct repeat sequences (repeat sequences contained in pre-crRNA) corresponding to Cas12f.4, Cas12f.5, and Cas12f.6 are shown in SEQ ID NOs: 7, 8, and 9, respectively. The mature direct repeat sequences (repeat sequences contained in mature crRNA) corresponding to Cas12f.4, Cas12f.5, and Cas12f.6 are shown in SEQ ID NOs: 13, 14, and 15, respectively.

### Example 2. Processing of Mature crRNA by Cas12f Gene

1. The double-stranded DNA molecule as shown in SEQ ID NO: 4 was artificially synthesized, and the double-stranded DNA molecule as shown in SEQ ID NO: 10 was artificially synthesized at the same time.
2. Connecting the double-stranded DNA molecule synthesized in step 1 with the prokaryotic expression vector pACYC-Duet-1 to obtain the recombinant plasmid pACYC-Duet-1+CRISPR/Cas12f.

The recombinant plasmid pACYC-Duet-1+CRISPR/Cas12f was sequenced. Sequencing results show that the recombinant plasmid pACYC-Duet-1+CRISPR/Cas12f contains the sequences as shown in SEQ ID NO: 4 and SEQ ID NO: 10, and expresses the Cas12f.4 protein as shown in SEQ ID NO: 1 and the Cas12f.4 prototype direct repeat sequence as shown in SEQ ID NO: 7. The recombinant plasmid pACYC-Duet-1+CRISPR/Cas12f was introduced into E. coli EC100 to obtain a recombinant bacteria, which was named EC100-CRISPR/Cas12f.
3. Taking a single clone of EC100-CRISPR/Cas12f, inoculating it into 100 mL LB liquid medium (containing 50 μg/mL ampicillin), culturing with shaking at 37° C. and 200 rpm for 12 h to obtain a culture broth.
4. Extracting bacterial RNA: transferring 1.5 mL of bacterial culture to a pre-cooled microcentrifuge tube and centrifuged at 6000×g for 5 minutes at 4° C. After centrifugation, discarding the supernatant, and resuspendings the cell pellet in 200 μL Max Bacterial Enhancement Reagent preheated to 95° C. Mixed by pipetting and mixed well, and incubated at 95° C. for 4 minutes. Adding 1 mL of TRIzol® Reagent to the lysate and mixed by pipetting and incubated at room temperature for 5 minutes. Adding 0.2 mL cold chloroform, shaking the tube by hand to mix for 15 seconds, and incubated at room temperature for 2-3 minutes. Centrifuged at 12,000×g for 15 minutes at 4° C. Taking 600 μL of supernatant in a new tube, adding 0.5 mL of cold isopropanol to precipitate RNA, mixed upside down, and incubated at room temperature for 10 minutes. Centrifuged at 15,000×g for 10 minutes at 4° C., discarding the supernatant, adding 1 mL of 75% ethanol, and for the vortex to mix. Centrifuged at 7500×g for 5 minutes at 4° C., discarding the supernatant, and for the air dry. Dissolving the RNA pellet in 50 μL RNase-free water and incubated at 60° C. for 10 minutes.
5. DNA digestion: 20 μg RNA was dissolved in 39.5 μL dH₂O, 65° C., 5 min. 5 min on ice, adding 0.5 μL RNAI, 5 μL buffer, 5 μL DNaseI, 37° C. for 45 min (50 μL system). Adding 50 μL dH₂O and adjusting the volume to 100 μL. After centrifuging the 2 mL Phase-Lock tube at 16000 g for 30 s, adding 100 μL of phenol: chloroform: isoamyl alcohol (25:24:1), 100 μL of digested RNA, shaked for 15 s, and centrifuged at 16000 g for 12 min at 15° C. Taking the supernatant into a new 1.5 mL centrifuge tube, adding the same volume of isopropanol 1/10 NaoAC as the supernatant, and reacted for 1 hour or −20° C. overnight. Centrifuged at 16000 g for 30 min at 4° C., and discarding the supernatant. Adding 350 μL of 75% ethanol to wash the pellet, centrifuged at 16000 g for 10 min at 4° C., and discarding the supernatant. Drying, and adding 20 μL RNase-free water at 65° C. for 5 min to dissolve the precipitate. Using NanoDrop to measure the concentration and running the gel.
6. 3′ dephosphorylation and 5′ phosphorylation: Adding water to ~20 μg of each digested RNA to 42.5 μL, at 90° C. for 2 min. Cooling on ice for 5 minutes. Adding 5 μL 10×T4 PNK buffer; 0.5 μL RNal, 2 μL T4 PNK (50 μL), at 37° C. for 6 h. Adding 1 μL T4 PNK, 1.25 μL (100 mM) ATP, 37° C. for 1 h. Adding 47.75 μL dH₂O and adjusting the volume to 100 μL. After centrifuging the 2 mL Phase-Lock tube at 16000 g for 30 s, adding 100 μL of phenol: chloroform: isoamyl alcohol (25:24:1), 100 μL of digested RNA, shaking for 15 s, and centrifuged at 16000 g for 12 min at 15° C. Taking the supernatant into a new 1.5 mL centrifuge tube, adding the same volume of isopropanol with the supernatant, the total volume of 1/10 NaoAC, and reacted for 1 hour or −20° C. overnight. Centrifuged at 16000 g for 30 min at 4° C., and discarding the supernatant. Adding 350 μL of 75% ethanol to wash the pellet, centrifuged at 16000 g for 10 min at 4° C., and discarding the supernatant. Drying, and adding 21 μL RNase-free water at 65° C. for 5 min to dissolve the precipitate, using NanoDrop to measure the concentration.
7. RNA monophosphorylation: 20 μL RNA, at 90° C. for 1 min, cooling on ice for 5 min. Adding 2 μL RNA 5′ Polphosphatase 10×Reaction buffer, 0.5 μL Inhibitor, 1 μL RNA 5′ Polphosphatase (20 Units), and adding RNase-free water to 20 μL, at 37° C. for 60 min. Adding 80 μL dH₂O and adjusting the volume to 100 μL. After centrifuging the 2 mL Phase-Lock tube at 16000 g for 30 s, adding 100 μL of phenol: chloroform: isoamyl alcohol (25:24:1), 100 μL of digested RNA, shaking for 15 s, and centrifuged at 16000 g for 12 min at 15° C. Taking the supernatant in a new 1.5 mL centrifuge tube, adding the same volume of isopropanol with the supernatant, the total volume of 1/10 NaoAC, and reacted for 1 hour or −20° C. overnight. Centrifuged at 16000 g for 30 min at 4° C., discarding the supernatant, adding 350 μL of 75% ethanol to wash the precipitate, centrifuged at 16000 g for 10 min at 4° C., discarding the supernatant. Drying, and adding 21 μL RNase-free water at 65° C. for 5 min to dissolve the precipitate, using NanoDrop to measure the concentration.
8. Preparation of cDNA library: 16.5 μL RNase-free water. 5 μL Poly (A) Polymerase 10×Reaction buffer. 5 μL 10 mM ATP. 1.5 μL RiboGuard RNase Inhibitor. 20 μL RNA Substrate. 2 μL Poly (A) Polymerase (4 Units). 50 μL of total volume at 37° C. for 20 minutes. Adding 50 μL dH₂O and adjusting the volume to 100 μL. After centrifuging the 2 mL Phase-Lock tube at 16000 g for 30 s, adding 100 μL of phenol:chloroform:isoamyl alcohol (25:24:1), 100 μL of digested RNA, shaking for 15 s, and centrifuged at 16000 g for 12 min. Taking the supernatant into a new 1.5 mL centrifuge tube, adding the same volume of isopropanol with the supernatant, the total volume of 1/10 NaoAC, and reacted for 1 hour or −20° C. overnight. Centrifuged at 16000 g for 30 min at 4° C., discarding the supernatant, drying it, and adding 11 μL RNase-free water at 65° C. for 5 min to dissolve the precipitate, and measuring the concentration with NanoDrop.
9. Adding the sequencing linker to the cDNA library and sending it to Beijing berrygenomics for sequencing.
10. Performing quality filtering on the original data to remove sequences with an average base quality value lower than 30. After removing the linker from the sequence, the RNA sequence from 25 nt to 50 nt was retained, and aligned to the reference sequence of the CRISPR array with bowtie.
11. Through comparison, we have found that the pre-crRNA of Cas12f.4 can be successfully processed into 45nt mature crRNA in E. coli, which consists of 23nt Repeat sequence and 19-22nt targeting sequence.
12. Using ViennaRNA and VARNA to predict and visualize the structure of mature crRNA. We have found that the 3′end of the Repeat sequence of crRNA can form an 8-base neck loop (FIG. 1).
13. After predicting the 23nt sequence of the 3′ end of the crRNA of Cas12f.5 and Cas12f.6, we have found a similar secondary structure (FIG. 1).

### Example 3. Identification of the PAM Domain of the Cas12f Gene

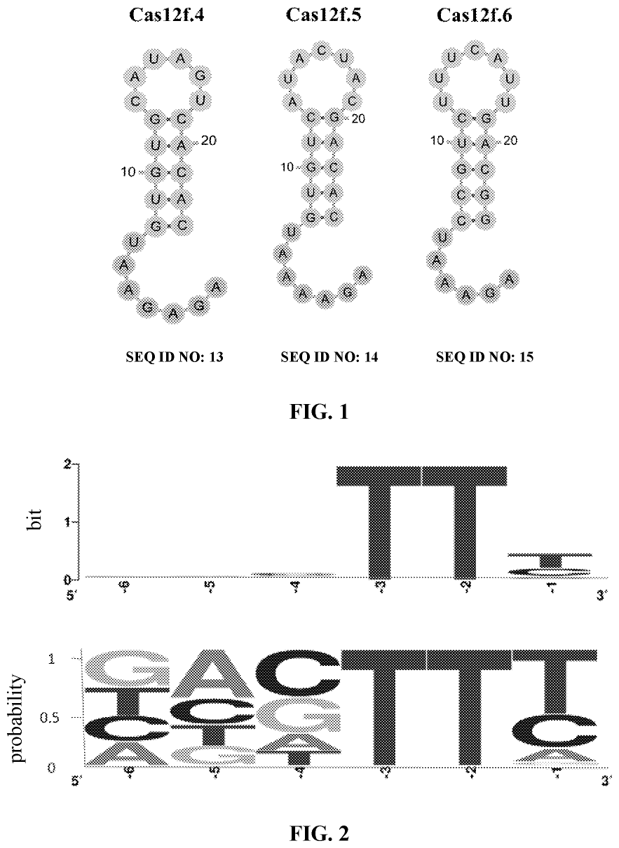

1. Constructing the recombinant plasmid pACYC-Duet-1+CRISPR/Cas12f and sequencing it. According to the sequencing results, the structure of the recombinant plasmid pACYC-Duet-1+CRISPR/Cas12f is described as follows: Replacing the small fragment between the recognition sequence of the restriction endonuclease Pml I and Kpn I of the vector pACYC-Duet-1 with the double-stranded sequence shown at positions 1 to 3713 from the 5′ end in the sequence as shown in SEQ ID NO: 4. The recombinant plasmid pACYC-Duet-1+CRISPR/Cas12f expresses the Cas12f.4 protein as shown in SEQ ID NO: 1 and the Cas12f guide RNA as shown in SEQ ID NO: 25.
2. The recombinant plasmid pACYC-Duet-1+CRISPR/Cas12f contains an expression cassette, and the nucleotide sequence of the expression cassette is shown in SEQ ID NO: 23. In the sequence as shown in SEQ ID NO: 23, positions 1 to 44 from the 5′ end are the nucleotide sequence of the pLacZ promoter, positions 45 to 3326 are the nucleotide sequence of the Cas12f.4 gene, and positions 3327 to 3412 are the nucleotide sequence of the terminator (used to terminate transcription). From the 5′ end, positions 3413 to 3452 are the nucleotide sequence of the J23 119 promoter, positions 3453 to 3,628 are the nucleotide sequence of the CRISPR array, and positions 3627 to 3713 are the nucleotide sequence of the rrnB-T1 terminator (used to terminate transcription).
3. The acquisition of the recombinant E. coli: the recombinant plasmid pACYC-Duet-1+CRISPR/Cas12f was introduced into E. coli EC100 to obtain recombinant E. coli, named EC100/pACYC-Duet-1+CRISPR/Cas12f. The recombinant plasmid pACYC-Duet-1 was introduced into E. coli EC100 to obtain a recombinant E. coli named EC100/pACYC-Duet-1.
4. Construction of the PAM library: the sequence shown in SEQ ID NO: 24 is artificially synthesized and connected to the pUC19 vector, wherein the sequence as shown in SEQ ID NO: 24 includes eight random bases at the 5′ end and the target sequence. Eight random bases were designed in front of the 5′ end of the target sequence of the PAM library to construct a plasmid library. The plasmids were transferred into Escherichia coli containing the Cas12f.4 locus and Escherichia coli without the Cas. 12f.4 locus, respectively. After treatment at 37° C. for 1 hour, we extracted the plasmid, and performed PCR amplification and sequencing on the sequence of the PAM region.
5. The acquisition of the PAM library domain: the number of occurrences of 65,536 combinations of PAM sequences in the experimental group and the control group were counted, and the number of PAM sequences in each group was used for normalization. For any PAM sequence, when the log 2 (normalized value of the control group/normalized value of the experimental group) is greater than 3.5, we deem that this PAM is significantly consumed. We obtained a total of 3,548 significantly consumed PAM sequences, all accounting for 5.41%. We used Weblogo to predict the significantly consumed PAM sequence and found that the PAM domain of Cas12f.4 was a strict 5′-TTN structure (FIG. 2), and almost 100% of the second and third bases in front of the target sequence were T, and the other positions can be any sequence. This is a more rigorous PAM recognition method than C2c1, which has been reported for the most rigorous PAM recognition.
6. Verification of the PAM library domain: Through the PAM library consumption experiment, we obtained the PAM domain of Cas12f.4. In order to verify the rigor of this domain, we set up 10 groups of PAM for in vivo experiments and sequenced Cas12f's editing activity on these PAMs. First, we integrated the 30 nt target and PAM sequence into the non-conserved position of the Kana gene-resistance of the plasmid, and then mixed it with the complex formed by CRSPR/Cas12f and guide RNA for 8 hours. By coating the plate and counting the number of colonies, we can judge the consumption activity of Cas1 2f on different PAM sequences. Through the experimental results, we can see that the CRISPR/Cas12f.4 system can only effectively edit the target sequence with 5′-TTA, 5′-TTT, 5′-TTC and 5′-TTG PAM, it has no editing activity on target sequences with 5′-TAT, 5′-TCT, 5′-TCG, 5′-ATT, 5′-CTT and 5′-GTT PAM, thus verifying the verifiability of the PAM domain recognition of Cas12f.4. By counting the colonies of different PAMs, we have found that the editing activity of the CRISPR/Cas12f.4 system on 5′-TTA, 5′-TTT and 5′-TTC is higher than that on 5′-TTG.

### Example 4. Cas12f.4, Cas12f.5, Cas12f.6 Cleavage in Human Cell Lines

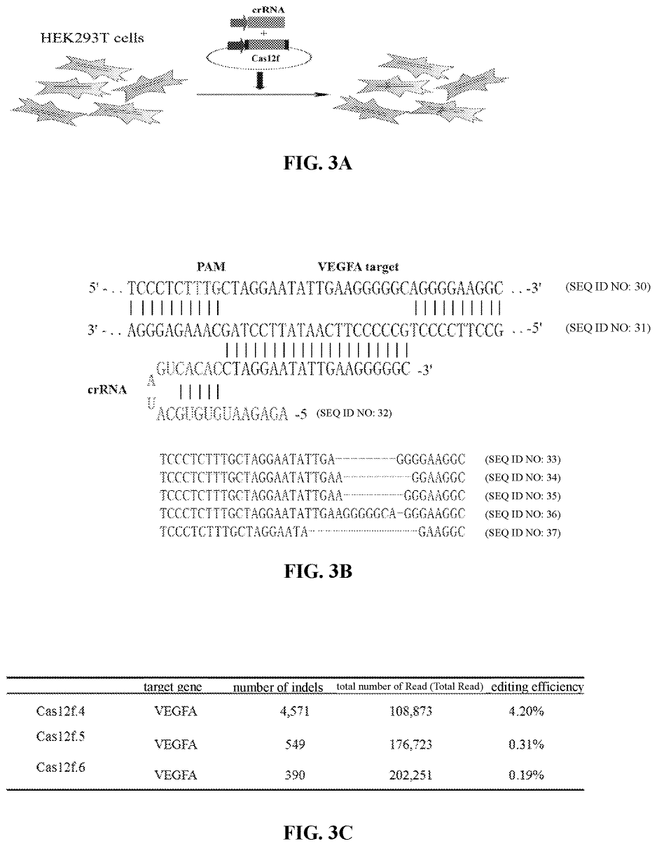

The eukaryotic expression vector containing the Cas12f.4 gene and the PCR product containing the U6 promoter and crRNA (SEQ ID NO: 25) sequence were transfected into a human HEK293T cell by liposome transfection (FIG. 3A), and incubated for 72 hours at 37 degrees Celsius with 5% carbon dioxide concentration. The DNA of total cells was extracted, and the 700 bp sequence containing the target site was amplified. The PCR products were constructed for next-generation sequencing library through Tn5, and the sequencing was completed by Beijing Annoroad Genomics Technology Co., Ltd. The sequencing results were compared to the VEGFA gene of the human genome, the cleavage method of Cas12f.4 to the target site was identified (FIG. 3B). The editing efficiency of CRISPR/Cas12f.4 system for VEGFA can reach 4.2%. The original sequencing data is shown in FIG. 3C.

The same method was used to detect the cleavage activity of Cas12f.5 and Cas12f.6 on VEGFA, and their crRNAs are shown in SEQ ID NO: 26 and SEQ ID NO: 27, respectively. The results in FIG. 3C show that the editing efficiency of CRISPR/Cas12f.5 and CRISPR/Cas12f.6 systems on VEGFA are 0.31% and 0.19%, respectively.

### Example 5. Cleavage of Cas12f.4 in a Maize Protoplast

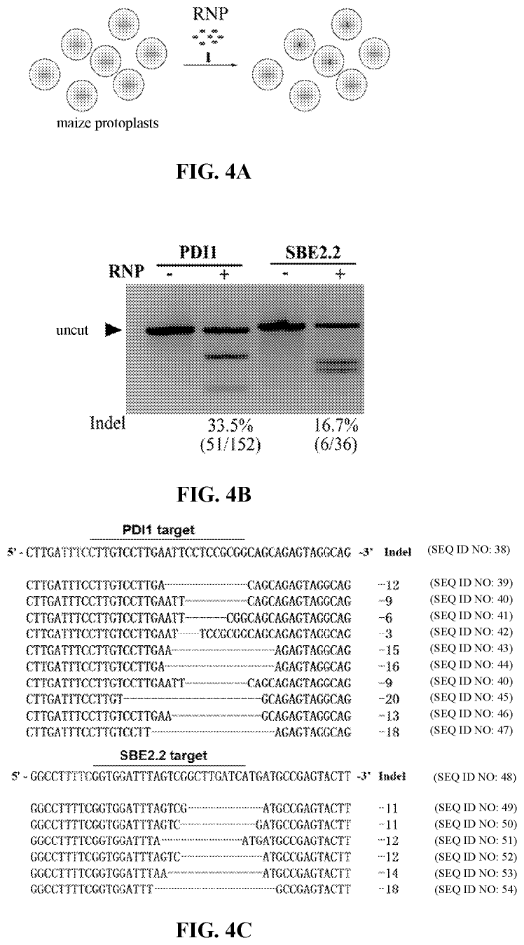

The purified Cas12f.4 protein (60 μg) and the guide RNA (120 μg) as shown in SEQ ID NO: 28 or 29 were mixed at 37 degrees Celsius to form a ribonucleoprotein complex (RNP), and then the CRISPR/Cas12f.4 RNP was transferred into a maize protoplast cell using PEG4000-mediated protoplast transformation, and cultured in the dark at 37 degrees Celsius for 24 hours (FIG. 4A). After the culture, the supernatant was discarded by centrifugation to collect the protoplasts, and the protoplast DNA was extracted. The DNA fragments of about 600 bp upstream and downstream of the target site were amplified. The DNA fragment containing the target site was subjected to T7 endonuclease digestion detection, and the result was shown in FIG. 4B. The CRISPR/Cas12f.4 system has a high-efficiency cleavage activity for PDI1 and SEB2.2. Connecting the DNA fragment containing the target site to the Blunt Simple vector, coating the plate, and using Thermo Fisher Scientific (China) Co., Ltd. to perform Sanger sequencing on the single clone, and comparing the sequencing results to the PDI1 and SEB2.2 genes in the maize group, the results are shown in FIGS. 4B-4C. The cleavage efficiency of Cas12f.4 on the target site is identified as 33.5% and 16.7%, respectively.

Although the specific embodiments of the present invention have been described in detail, those skilled in the art will understand that various modifications and changes can be made to the details according to all the teachings that have been published, and these changes are within the protection scope of the present invention. All of the present invention is given by the appended claims and any equivalents thereof.

What is claimed is:

1. An engineered, non-naturally occurring clustered regularly interspaced short palindromic repeats (CRISPR)-CRISPR-associated (Cas) system, comprising:
(a) one or more guide RNAs or polynucleotides encoding the guide RNAs, wherein the guide RNA comprises a direct repeat sequence and a targeting sequence; and
(b) a CRISPR-Cas effector protein or one or more nucleotide sequences encoding the CRISPR-Cas effector protein, wherein the CRISPR-Cas effector protein comprises an amino acid sequence having at least 90%, at least 95%, at least 98%, or at least 99% sequence identity compared to SEQ ID NO: 1 or SEQ ID NO: 2, or having 1, 2, 3, 4, 5, 6, 7, 8, 9, or 10 amino acid substitutions, deletions, or additions compared to SEQ ID NO: 1 or SEQ ID NO: 2; or wherein the CRISPR-Cas effector protein is as set forth in SEQ ID NO: 1 or SEQ ID NO: 2;
wherein the CRISPR-Cas effector protein binds to the one or more guide RNAs to form a complex, the targeting sequence hybridizes to a target sequence of a target nucleic acid, and the target sequence is located at a 3′ end of a protospacer adjacent motif (PAM).

2. The engineered, non-naturally occurring CRISPR-Cas system of claim 1, wherein the engineered, non-naturally occurring CRISPR-Cas system does not comprise trans-activating crRNA (tracrRNA).

3. The engineered, non-naturally occurring CRISPR-Cas system of claim 1, wherein the targeting sequence is located at a 3′ end of the direct repeat sequence, or the targeting sequence comprises a complementary sequence of the target sequence.

4. The engineered, non-naturally occurring CRISPR-Cas system of claim 1, wherein
the direct repeat sequence comprises a polynucleotide sequence having at least 90% sequence identity compared to SEQ ID NO: 7 or SEQ ID NO: 13, or the direct repeat sequence is as set forth in SEQ ID NO: 7 or SEQ ID NO: 13; or
the direct repeat sequence comprises a polynucleotide sequence having at least 90% sequence identity to SEQ ID NO: 8 or SEQ ID NO: 14, or the direct repeat sequence is as set forth in SEQ ID NO: 8 or SEQ ID NO: 14.

5. The engineered, non-naturally occurring CRISPR-Cas system of claim 1, wherein the CRISPR-Cas effector protein comprises a modified portion, an additional protein, or an additional polypeptide, the modified portion, the additional protein, or the additional polypeptide is selected from a protein, a polypeptide, a detectable label, an epitope tag, a reporter gene sequence, a nuclear localization signal (NLS) sequence, a targeting moiety, a transcription activation domain, a transcription repression domain, a nuclease domain, a domain having an activity selected from nucleotide deaminase activity, methylase activity, demethylase activity, transcription activation activity, transcription inhibition activity, transcription release factor activity, histone modification activity, nuclease activity, single-stranded RNA cleavage activity, double-stranded RNA cleavage activity, single-stranded DNA cleavage activity, double-stranded DNA cleavage activity, nucleic acid binding activity, and any combination thereof.

6. The engineered, non-naturally occurring CRISPR-Cas system of claim 5, wherein the transcription activation domain is VP64, the transcription repression domain is KRAB domain or SID domain, and the nuclease domain is FokI.

7. The engineered, non-naturally occurring CRISPR-Cas system of claim 5, wherein the modified portion, the additional protein, or the additional polypeptide is linked to an N-terminus or a C-terminus of the CRISPR-Cas effector protein via a linker.

8. The engineered, non-naturally occurring CRISPR-Cas system of claim 5, wherein the CRISPR-Cas effector protein comprises one or more nuclear localization signals (NLS), wherein an NLS sequence is as set forth in SEQ ID NO: 19, and the CRISPR-Cas effector protein has the amino acid sequence as set forth in SEQ ID NO: 20 or SEQ ID NO: 21.

9. An isolated nucleic acid molecule, comprising: (i) a polynucleotide sequence encoding the CRISPR-Cas effector protein recited in the engineered, non-naturally occurring CRISPR-Cas system of claim 1; and/or (ii) a polynucleotide sequence encoding the one or more guide RNAs recited in the engineered, non-naturally occurring CRISPR-Cas system of claim 1; wherein for example, the polynucleotide sequence of (i), the polynucleotide sequence of (ii), or each of the polynucleotide sequences of (i) and (ii) is codon optimized for expression in a prokaryotic cell or a eukaryotic cell.

10. A vector system, comprising:
(a) a first regulatory element operably linked to a polynucleotide sequence encoding a guide RNA, the guide RNA comprising a targeting sequence linked to a direct repeat sequence, wherein the targeting sequence is capable of hybridizing to a target sequence; and
(b) a second regulatory element operably linked to a nucleotide sequence encoding a CRISPR-Cas effector protein, wherein the CRISPR-Cas effector protein comprises an amino acid sequence having at least 90%, at least 95%, at least 98%, or at least 99% sequence identity compared to SEQ ID NO: 1, SEQ ID NO: 2, SEQ ID NO: 20, or SEQ ID NO: 21, or having 1, 2, 3, 4, 5, 6, 7, 8, 9, or 10 amino acid substitutions, deletions, or additions compared to SEQ ID NO: 1, SEQ ID NO: 2, SEQ ID NO: 20, or SEQ ID NO: 21, and the CRISPR-Cas effector protein is capable of forming a CRISPR complex with the guide RNA in the absence of tracrRNA;
wherein the first regulatory element and the second regulatory element are located on a same vector or on different vectors of the vector system;
wherein, when transcribed, the targeting sequence hybridizes to the target sequence, the target sequence is located at a 3′ end of a protospacer adjacent motif (PAM), and the guide RNA forms a complex with the CRISPR-Cas effector protein.

11. The vector system of claim 10, wherein the CRISPR-Cas effector protein comprises the amino acid sequence set forth in SEQ ID NO: 1, SEQ ID NO: 2, SEQ ID NO: 20, or SEQ ID NO: 21.

12. The vector system of claim 10, wherein
each of the first regulatory element and the second regulatory element is a promoter,
at least one of the first regulatory element and the second regulatory element is non-naturally occurring or modified,
the targeting sequence is linked to a 3′ end of the direct repeat sequence,
the targeting sequence comprises a complementary sequence of the target sequence,
when the target sequence is DNA, the target sequence is located at the 3′ end of the PAM, and the PAM has a sequence shown by 5′-TTN, wherein Nis selected from A, G, T, and C; when the target sequence is RNA, the target sequence does not have PAM domain restrictions,
the target sequence is a DNA or RNA sequence derived from a prokaryotic cell or a eukaryotic cell, or wherein the target sequence is a non-naturally occurring DNA or RNA sequence,
the target sequence is present in a cell, and
the CRISPR-Cas effector protein is linked to one or more NLS sequences, the one or more NLS sequences are linked to an N-terminus or C-terminus of the CRISPR-Cas effector protein, or the one or more NLS sequences are fused to the N-terminus or C-terminus of the CRISPR-Cas effector protein.

13. A cell, cell line, or progeny thereof, comprising the engineered, non-naturally occurring CRISPR-Cas system of claim 1; wherein the cell is a eukaryotic cell or a prokaryotic cell, the eukaryotic cell is an animal cell or a plant cell, and wherein the animal cell is a mammalian stem cell, or the cell line is a mammalian stem cell line.

14. A delivery composition, comprising:
(i) a delivery vehicle; and
(ii) the engineered, non-naturally occurring CRISPR-Cas system of claim 1;
wherein the delivery vehicle is selected from a lipid particle, a sugar particle, a metal particle, a protein particle, a liposome, an exosome, a microvesicle, a gene gun, a viral vector, polyethylene glycol, or Agrobacterium.

15. The delivery composition of claim 14, wherein the viral vector comprises replication-defective retrovirus, lentivirus, adenovirus, or adeno-associated virus;
wherein the Agrobacterium comprises Agrobacterium tumefaciens or Agrobacterium rhizogenes.

16. A method of preparing a formulation, comprising using the engineered, non-naturally occurring CRISPR-Cas system of claim 1, wherein the formulation is configured for: (i) isolated gene or genome editing; (ii) a detection of an isolated single-stranded DNA; (iii) editing the target sequence in a target locus to modify an organism or non-human organism; (iv) treating a disease caused by defects in the target sequence in the target locus.

17. A method of modifying a target gene, comprising contacting the engineered, non-naturally occurring CRISPR-Cas system of claim 1 with the target gene, or delivering the engineered, non-naturally occurring CRISPR-Cas system to a cell containing the target gene.

18. The method of modifying the target gene of claim 17, wherein
the target gene comprises a target sequence,
the target gene is present in a cell selected from a prokaryotic cell, a eukaryotic cell, a mammalian cell, or a plant cell, and/or the target gene is present in an in vitro nucleic acid molecule, wherein the in vitro nucleic acid molecule is a plasmid, and/or
the modifying refers to generating a break in the target sequence, wherein the break comprises a double-strand break in DNA or a single-strand break in RNA; wherein the modifying further comprises deleting, substituting, or inserting a sequence at the break, furthermore,
wherein the CRISPR-Cas system is delivered to the cell as one or more polynucleotide molecules.

19. A method of altering an expression of a gene product or regulating a gene expression, comprising contacting the engineered, non-naturally occurring CRISPR-Cas system of claim 1 with a target gene, or delivering the engineered, non-naturally occurring CRISPR-Cas system to a cell containing the target gene; wherein
the target gene is present in a cell selected from a prokaryotic cell, a eukaryotic cell, a mammalian cell, and a plant cell, and/or
the target gene is present in an in vitro nucleic acid molecule, wherein the in vitro nucleic acid molecule is a plasmid, and/or
an alteration of the expression of the gene product refers to an enhancement, reduction, or demethylation, wherein the gene product is a protein, and/or
the method is used to alter one or more target sequences in the target gene or a nucleic acid molecule encoding a target gene product to modify a cell, cell line, or organism, furthermore,
wherein the CRISPR-Cas system is delivered to the cell as one or more polynucleotide molecules.

20. A method of producing a plant having a modified trait of interest encoded by a gene of interest, comprising contacting a plant cell with the engineered, non-naturally occurring CRISPR-Cas system of claim 1, thereby modifying or introducing the gene of interest, and regenerating the plant from the plant cell.

21. A method of producing a plant having a modified trait of interest encoded by a gene of interest, comprising subjecting the plant cell to the method of claim 17, thereby modifying or introducing the gene of interest, and regenerating the plant from the plant cell.

22. A method of identifying a trait of interest in a plant, the trait of interest being encoded by a gene of interest, the method comprising contacting a plant cell with the engineered, non-naturally occurring CRISPR-Cas system of claim 1, thereby identifying the gene of interest.

23. The method of identifying the trait of interest in the plant of claim 22, further comprising introducing the gene of interest identified into a plant cell, plant cell line, or plant germplasm, and generating the plant therefrom, thereby causing the plant to contain the gene of interest.

24. The method of identifying the trait of interest in the plant of claim 23, wherein the plant exhibits the trait of interest.

25. A method of identifying a trait of interest in a plant, the trait of interest being encoded by a gene of interest, the method comprising subjecting the plant cell to the method of claim 17, thereby identifying the gene of interest.

26. A cell or progeny thereof obtained by the method of claim 17, wherein the cell comprises a modification not present in a wild type of the cell.

27. A cell product of the cell or progeny thereof of claim 26, wherein the cell comprises a modification not present in a wild type of the cell.

28. A cell or progeny thereof obtained by the method of claim 19, wherein the cell comprises a modification not present in a wild type of the cell.

29. A cell product of the cell or progeny thereof of claim 28, wherein the cell comprises a modification not present in a wild type of the cell.

30. A method of preparing a formulation, comprising using a CRISPR-Cas effector protein, wherein the formulation is configured for: (i) isolated gene or genome editing; (ii) a detection of an isolated single-stranded DNA; (iii) editing the target sequence in a target locus to modify an organism or non-human organism; (iv) treating a disease caused by defects in the target sequence in the target locus; wherein the CRISPR-Cas effector protein comprises an amino acid sequence having at least 90%, at least 95%, at least 98%, or at least 99% sequence identity compared to SEQ ID NO: 1 or SEQ ID NO: 2, or having 1, 2, 3, 4, 5, 6, 7, 8, 9, or 10 amino acid substitutions, deletions, or additions compared to SEQ ID NO: 1 or SEQ ID NO: 2.

31. The method of preparing the formulation of claim 30, wherein the CRISPR-Cas effector protein is as set forth in SEQ ID NO: 1 or SEQ ID NO: 2.

32. A method of nucleic acid editing, comprising contacting a CRISPR-Cas effector protein or a delivery composition with the target gene, or delivering the CRISPR-Cas effector protein or the delivery composition to a cell containing the target gene; furthermore,
wherein the nucleic acid editing comprises gene or genome editing, and the gene or genome editing comprises modifying a gene, knocking out a gene, gene base substitution, a altering an expression of a gene product or regulating a gene expression, repairing a mutation, and/or inserting a polynucleotide;
wherein the CRISPR-Cas effector protein comprises an amino acid sequence having at least 90%, at least 95%, at least 98%, or at least 99% sequence identity compared to SEQ ID NO: 1 or SEQ ID NO: 2, or having 1, 2, 3, 4, 5, 6, 7, 8, 9, or 10 amino acid substitutions, deletions, or additions compared to SEQ ID NO: 1 or SEQ ID NO: 2.

33. The method of nucleic acid editing of claim 32, wherein the delivery composition comprises a delivery vehicle and the CRISPR-Cas effector protein comprising the amino acid sequence set forth in SEQ ID NO: 1 or SEQ ID NO: 2.

34. The method of nucleic acid editing of claim 32, wherein the target gene is present in a cell selected from a prokaryotic cell, a eukaryotic cell, a mammalian cell, and a plant cell, and/or
the target gene is present in an in vitro nucleic acid molecule, wherein the in vitro nucleic acid molecule is a plasmid, and/or
an alteration of the expression of the gene product refers to an enhancement, reduction, or demethylation, wherein the gene product is a protein, and/or
the method is used to alter one or more target sequences in the target gene or a nucleic acid molecule encoding a target gene product to modify a cell, cell line, or organism.

35. A method of producing a plant having a modified trait of interest encoded by a gene of interest, comprising contacting a plant cell with a CRISPR-Cas effector protein or a delivery composition, thereby modifying or introducing the gene of interest, and regenerating the plant from the plant cell, wherein the delivery composition comprises a delivery vehicle and the CRISPR-Cas effector protein, wherein the CRISPR-Cas effector protein comprises an amino acid sequence having at least 90%, at least 95%, at least 98%, or at least 99% sequence identity compared to SEQ ID NO: 1 or SEQ ID NO: 2, or having 1, 2, 3, 4, 5, 6, 7, 8, 9, or 10 amino acid substitutions, deletions, or additions compared to SEQ ID NO: 1 or SEQ ID NO: 2.

36. The method of producing the plant having the modified trait of interest encoded by the gene of interest of claim 35, wherein the CRISPR-Cas effector protein comprising the amino acid sequence set forth in SEQ ID NO: 1 or SEQ ID NO: 2.

37. A method of identifying a trait of interest in a plant, the trait of interest being encoded by a gene of interest, the method comprising contacting a plant cell with a CRISPR-Cas effector protein or a delivery composition, thereby identifying the gene of interest; wherein the delivery composition comprises a delivery vehicle and the CRISPR-Cas effector protein, wherein the CRISPR-Cas effector protein comprises an amino acid sequence having at least 90%, at least 95%, at least 98%, or at least 99% sequence identity compared to SEQ ID NO: 1 or SEQ ID NO: 2, or having 1, 2, 3, 4, 5, 6, 7, 8, 9, or 10 amino acid substitutions, deletions, or additions compared to SEQ ID NO: 1 or SEQ ID NO: 2.

38. The method of identifying a trait of interest in a plant, the trait of interest being encoded by a gene of interest of claim 37, wherein the CRISPR-Cas effector protein comprising the amino acid sequence set forth in SEQ ID NO: 1 or SEQ ID NO: 2.

39. The method of nucleic acid editing of claim 32, wherein the delivery vehicle is selected from a lipid particle, a sugar particle, a metal particle, a protein particle, a liposome, an exosome, a microvesicle, a gene gun, a viral vector, polyethylene glycol, or Agrobacterium.

40. The method of nucleic acid editing of claim 39, wherein the viral vector comprises replication-defective retrovirus, lentivirus, adenovirus, or adeno-associated virus, wherein the Agrobacterium comprises Agrobacterium tumefaciens or Agrobacterium rhizogenes.

41. The method of producing the plant having the modified trait of interest encoded by the gene of interest of claim 35, wherein the delivery vehicle is selected from a lipid particle, a sugar particle, a metal particle, a protein particle, a liposome, an exosome, a microvesicle, a gene gun, a viral vector, polyethylene glycol, or Agrobacterium.

42. The method of producing the plant having the modified trait of interest encoded by the gene of interest of claim 41, wherein the viral vector comprises replication-defective retrovirus, lentivirus, adenovirus, or adeno-associated virus, wherein the Agrobacterium comprises Agrobacterium tumefaciens or Agrobacterium rhizogenes.

43. The method of identifying the trait of interest in the plant of claim 37, wherein the delivery vehicle is selected from a lipid particle, a sugar particle, a metal particle, a protein particle, a liposome, an exosome, a microvesicle, a gene gun, a viral vector, polyethylene glycol, or Agrobacterium.

44. The method of identifying the trait of interest in the plant of claim 43, wherein the viral vector comprises replication-defective retrovirus, lentivirus, adenovirus, or adeno-associated virus, wherein the Agrobacterium comprises Agrobacterium tumefaciens or Agrobacterium rhizogenes.


SEQUENCE LISTING (54 sequences)

SEQ ID NO: 1 - protein, 1045 aa, synthetic construct
  REGION 1..1045: note = amino acid sequence of Cas12f.4
MKKVEVSRPYQSLLLPNHRKFKYLDETWNAYKSVKSLLHRFLVCAYGAVPFNKFVEVVEKVDNDQLVLAFAVRLFRLVPVESTSFAKVDKANLAKSLANHLPVGTAIPANVQSYFDSNFDPKKYMWIDCAWEADRLAREMGLSASQFSEYATTMLWEDWLPLNKDDVNGWGSVSGLFGEGKKEDRQQKVKMLNNLLNGIKKNPPKDYTQYLKILLNAFDAKSHKEAVKNYKGDSTGRTASYLSEKSGEITELMLEQLMSNIQRDIGDKQKEISLPKKDVVKKYLESESGVPYDQNLWSQAYRNAASSIKKTDTRNFNSTLEKFKNEVELRGLLSEGDDVEILRSKFFSSEFHKTPDKFVIKPEHIGFNNKYNVVAELYKLKAEATDFESAFATVKDEFEEKGIKHPIKNILEYIWNNEVPVEKWGRVARFNQSEEKLLRIKANPTVECNQGMTFGNSAMVGEVLRSNYVSKKGALVSGEHGGRLIGQNNMIWLEMRLLNKGKWETHHVPTHNMKFFEEVHAYNPSLADSVNVRNRLYRSEDYTQLPSSITDGLKGNPKAKLLKRQHCALNNMTANVLNPKLSFTINKKNDDYTVIIVHSVEVSKPRREVLVGDYLVGMDQNQTASNTYAVMQVVKPKSTDAIPFRNMWVRFVESGSIESRTLNSRGEYVDQLNHDGVDLFEIGDTEWVDSARKFFNKLGVKHKDGTLVDLSTAPRKAYAFNNFYFKTMLNHLRSNEVDLTLLRNEILRVANGRFSPMRLGSLSWTTLKALGSFKSLVLSYFDRLGAKEMVDKEAKDKSLFDLLVAINNKRSNKREERTSRIASSLMTVAQKYKVDNAVVHVVVEGNLSSTDRSASKAHNRNTMDWCSRAVVKKLEDMCNLYGFNIKGVPAFYTSHQDPLVHRADYDDPKPALRCRYSSYSRADFSKWGQNALAAVVRWASNKKSNTCYKVGAVEFLKQHGLFADKKLTVEQFLSKVKDEEILIPRRGGRVFLTTHRLLAESTFVYLNGVKYHSCNADEVAAVNICLNDWVIPCKKKMKEESSASG

SEQ ID NO: 2 - protein, 1079 aa, synthetic construct
  REGION 1..1079: note = amino acid sequence of Cas12f.5
MPKQKDLPYSCLSYLQPNERKLKLLNNTYDHLTHGSKIMFDTLIALMGGINPKMDVISENKDSEIKNNRDPQTMCATIWFRPMKSKRINKVWSPKQLKEQFLKYYQEYEADVKINDMVEAYFDSPLGENYVWVDCRKKYKQLVKELASIAKTTEANLKEDLDCDLECLFRPSEKKMKLYGSNKSWAIISNLFGEGDKEDRSKKIKILTKAIQILTESNPESYADVQKAFLAAADIDDPKKFHTQEIWGNGSPGNIVKMARGDFLGKEFDCEKILEKINDVLKEKTLDFDLKVRLSFKEYLISKIGHYYQNSWSEMINSAFADIISKNTRNVNFAKEKVQLQKTLSETSNAKVELLTDFFKSDFFLGDDKFDIAPHNLGGANGIKFFYDFCKKNEDQYFLEELLLEAAIEESVAEAKSKSLKEPHKDLLRYVFSIRKETTFEELRDAAKYIQTHKRIKNMSVHPTVKSDIGFNVTSGSALVGHVVSPSKKINGRIAGESGFIWICMKLWEGGDKWIEHHIPFTDTRFYEQIYKYNPDSKLEPVVLRTKRYGVDLTKFNLPPMKTDLKHVAPKEKNKHNYVKVQRRLQRLNHPDVPNTIWPKSNIGFTIRRKNGKYILNVVHKLPKNKVKKSVKPKFGDILIGVDQNQTTNHTCSIYKVVKKNTKEALLVPESDFYLKKIETIKVTSFTKARYNSEPIDQLHYEGISVDNEVFKNWCKDREQFVDSLSIKEFKNEFKRIKNKNENLYSFNADYLWLLKRIISGKLNKKKFDVSVFEKSIRNEILAMCSKEGLGPLRVSSLSSNSLKSIGFLKSAICSFISIALNRKGIEDKTDVQKNKIDPELFDLIGKIEQKRVNKRMEKTRRNADFILTMAVDYQKSSQKNVFLFCEGNLETAKTGNSKKRNSANVDWCSRKLFDFLKEKSLRHGIYFHAVTPHYTSHQDPFEYHPSNKVMLPRFAKFDKNNPIQDWAEKKYLGFANSDPESGTALYYKKGVENFFAHYQKGFKEKVELAEMKNVLNSNLKNGNLEHVFCPIRGGRYYLSTHPVTSDAKPFEFNGRKCYICDSDEVAATNIMLIGLFYV

SEQ ID NO: 3 - protein, 1054 aa, synthetic construct
  REGION 1..1054: note = amino acid sequence of Cas12f.6
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 4 - DNA, 3138 bp, synthetic construct
  misc_feature 1..3138: note = encoding nucleic acid sequence of Cas12f.4
  source 1..3138: mol_type = other DNA
atgaagaaggtcgaggtgtcgcggccataccagagcctgctcctgccaaaccaccggaagttcaagtacctcgacgagacctggaatgcgtacaagtccgttaagagcctgctccaccgcttcctggtgtgcgcttacggcgctgttcccttcaacaagttcgtggaggttgtcgagaaggttgataacgaccagctcgtgctggctttcgcggtgcgcctcttccgcctggtccccgtggagagcacctctttcgccaaggttgacaaggccaatctggcgaagtccctggccaatcacctgcctgtgggcacagccattcctgccaatgttcagtcctacttcgattcaaatttcgaccccaagaagtacatgtggatcgactgcgcgtgggaggctgatcgcctggctcgggagatgggcctgagcgcgagccagttctctgagtacgcgactacaatgctctgggaggactggctgcccctcaataaggatgatgtgaacggctgggggtccgtgtcggggctcttcggcgagggcaagaaggaggaccggcagcagaaggtgaagatgctgaataacctgctgaatggcatcaagaagaatccgcccaaggattacacccagtacctgaagatcctcctgaacgcgttcgacgcgaagtcgcacaaggaggctgttaagaactacaagggggactctacggggcgcaccgcgtcttacctgtcagagaagtctggcgagatcacagagctgatgctcgagcagctgatgtcaaacatccagagggatattggcgacaagcagaaggagatctccctgccgaagaaggacgtggtcaagaagtacctcgagtcagagtccggcgtcccatacgatcagaacctgtggtcccaggcctaccgcaacgctgccagctcgatcaagaagactgatacgcggaacttcaactccactctcgagaagttcaagaatgaggtggagctgaggggcctgctgagcgagggcgacgacgttgagatcctgaggtctaagttcttcagcagcgagttccacaagacccctgataagttcgttattaagccagagcatattgggttcaacaataagtacaatgtcgttgccgagctgtacaagctcaaggctgaggcgaccgatttcgagagcgctttcgccacagtcaaggatgagttcgaggagaagggcatcaagcacccaatcaagaacatcctcgagtacatctggaataacgaggtgcccgtcgagaagtggggccgggttgcccgcttcaaccagtccgaggagaagctcctccggattaaggccaaccccacggtggagtgcaaccagggcatgaccttcggcaattccgcgatggttggcgaggtcctcaggtccaactacgtctctaagaagggcgcgctggtgtccggcgagcacggcgggcgcctgatcggccagaacaatatgatctggctggagatgcggctgctcaacaaggggaagtgggagacccaccacgttccaacccataacatgaagttcttcgaggaggtgcatgcctacaatccctctctggcggattctgttaacgtgcggaatcggctgtaccgctcagaggactacacccagctgccttcaagcattaccgacgggctgaagggcaatccgaaggcgaagctcctgaagcgccagcactgcgctctgaacaatatgacagctaatgttctcaatcctaagctgagcttcacgatcaacaagaagaacgatgattacacggtcatcattgtccatagcgttgaggtctcgaagcctcggagggaggtgctcgttggcgattacctcgtgggcatggaccagaatcagacagcgtctaatacatacgccgtcatgcaggtcgtcaagccgaagtctacagatgcgatcccgttccgcaacatgtgggtgcggttcgtggagtcagggtctatcgagtcccggaccctcaacagccgcggggagtatgttgatcagctgaatcatgacggcgtggacctcttcgagatcggcgatacggagtgggtggactccgcgcggaagttcttcaataagctcggcgttaagcacaaggatggcacactggttgatctgtctacggcgccccggaaggcctacgctttcaacaacttctacttcaagaccatgctgaatcatctccggagcaatgaggttgacctgacgctcctgcgcaatgagatcctccgggttgccaatgggcggttctccccgatgcgcctcggctcgctctcctggactactctcaaggccctgggctcgttcaagtccctggtgctgtcgtacttcgaccggctgggcgccaaggagatggtcgacaaggaggctaaggataagtctctcttcgacctcctcgtggctatcaacaacaagcgctctaataagcgcgaggagcggacttcccggattgcctccagcctcatgactgtggcgcagaagtacaaggttgataacgctgtggtccatgtggtcgtcgaggggaatctctccagcacggacaggagcgcgtcaaaggcccataatcggaacactatggattggtgctctagggccgtggtgaagaagctggaggacatgtgcaatctctacggcttcaatatcaagggcgtcccagccttctacacatcccaccaggacccgctcgtccaccgcgccgactacgatgaccctaagccggcgctcaggtgccgctactcctcgtactcaagggcggacttcagcaagtgggggcagaacgctctcgcggcggtggttcgctgggcgtctaataagaagtccaacacctgctacaaggtcggggccgtggagttcctcaagcagcacggcctcttcgcggacaagaagctgacagtcgagcagttcctctcgaaggtgaaggacgaggagatcctcattccccgcaggggcgggagggtgttcctcacaactcaccggctcctggcggagtccactttcgtgtacctgaacggcgttaagtaccattcatgcaacgccgatgaggtggcggctgttaacatctgcctgaatgactgggttatcccgtgcaagaagaagatgaaggaggagtcaagcgcgtccgggtag

SEQ ID NO: 5 - DNA, 3240 bp, synthetic construct
  misc_feature 1..3240: note = encoding nucleic acid sequence of Cas12f.5
  source 1..3240: mol_type = other DNA
atgccgaagcagaaggacctcccctactcctgcctgagctacctccagccgaacgagaggaagctcaagctcctgaacaacacctacgaccacctgacgcacggcagcaagatcatgttcgacaccctgatcgcgctcatgggcggcatcaaccccaagatggacgtgatctccgagaacaaggacagcgagatcaagaacaaccgcgacccgcagaccatgtgcgccacgatctggttccgccccatgaagtccaagaggatcaacaaggtctggagcccgaagcagctcaaggagcagttcctgaagtactaccaggagtacgaggcggacgtgaagatcaacgacatggtcgaggcctacttcgactccccgctcggcgagaactacgtgtgggtcgactgcaggaagaagtacaagcagctggtgaaggagctggcctccatcgccaagaccacggaggccaacctgaaggaggacctcgactgcgacctggagtgcctcttccgccccagcgagaagaagatgaagctctacggctccaacaagagctgggcgatcatctccaacctgttcggcgagggcgacaaggaggacaggagcaagaagatcaagatcctcaccaaggccatccagatcctgacggagtccaaccccgagagctacgccgacgtgcagaaggccttcctcgctgccgcggacatcgacgacccgaagaagttccacacgcaggagatctggggcaacggcagccccggcaacatcgtcaagatggcccgcggcgacttcctgggcaaggagttcgactgcgagaagatcctcgagaagatcaacgacgtgctgaaggagaagacgctggacttcgacctcaaggtcaggctgtccttcaaggagtacctcatcagcaagatcggccactactaccagaactcctggagcgagatgatcaactccgcgttcgccgacatcatcagcaagaacacccgcaacgtgaacttcgcgaaggagaaggtccagctccagaagaccctgtccgagacgagcaacgccaaggtggagctgctgacggacttcttcaagtccgacttcttcctcggcgacgacaagttcgacatcgcgccgcacaacctgggcggcgccaacggcatcaagttcttctacgatttctgcaagaagaacgaggatcagtacttcctcgaggagctgctgctcgaggccgctatcgaggagtcggtggccgaggccaagtccaagagcctgaaggagccccacaaggacctgctccgctacgtcttcagcattaggaaggagacgaccttcgaggagctgagggacgccgccaagtacatccagacccacaagaggatcaagaacatgtccgtgcacccgaccgtcaagagcgacatcggcttcaacgtgacgtccggcagcgcgctcgtcggccacgtcgtgtcccccagcaagaagatcaacggcaggatcgcgggcgagagcggcttcatctggatctgcatgaagctgtgggagggcggcgacaagtggatcgagcaccacatcccgttcaccgacacgaggttctacgagcagatctacaagtacaacccggactccaagctcgagcccgtggtcctgcgcaccaagcgctacggcgtggacctcacgaagttcaacctgccgcccatgaagacggacctcaagcacgtcgccccgaaggagaagaacaagcacaactacgtgaaggtgcagaggcggctccagaggctcaaccaccccgacgtgcccaacaccatctggcccaagagcaacatcggcttcacgatccgcaggaagaacggcaagtacatcctcaacgtggtccacaagctgccgaagaacaaggtgaagaagtccgtcaagcccaagttcggcgacatcctgatcggcgtcgaccagaaccagaccacgaaccacacctgctccatctacaaggtggtcaagaagaacaccaaggaggcgctgctcgtgccggagagcgacttctacctcaagaagatcgagaccatcaaggtcacctccttcacgaaggccaggtacaacagcgagcccatcgaccagctgcactacgagggcatctccgtggacaacgaggtcttcaagaactggtgcaaggaccgcgagcagttcgtggactccctcagcatcaaggagttcaagaacgagttcaagaggatcaagaacaagaacgagaacctctactccttcaacgcggactacctgtggctgctcaagcgcatcatcagcggcaagctcaacaagaagaagttcgacgtgtccgtcttcgagaagagcatccgcaacgagatcctggctatgtgctccaaggagggcctgggccccctcagggtctccagcctctccagcaactccctgaagagcatcggcttcctcaagtccgcgatctgctccttcatcagcatcgccctgaacaggaagggcatcgaggacaagaccgacgtgcagaagaacaagatcgaccccgagctgttcgacctcatcggcaagatcgagcagaagcgcgtcaacaagaggatggagaagacccgcaggaacgcggacttcatcctcacgatggccgtggactaccagaagtccagccagaagaacgtcttcctcttctgcgagggcaacctggagaccgcgaagacgggcaactccaagaagcgcaacagcgccaacgtggactggtgctccaggaagctcttcgacttcctgaaggagaagagcctccgccacggcatctacttccacgcggtgaccccgcactacacgtcccaccaggacccgttcgagtaccaccccagcaacaaggtcatgctgccgaggttcgccaagttcgacaagaacaaccccatccaggactgggcggagaagaagtacctcggcttcgccaactccgaccccgagtcgggcaccgcgctgtactacaagaagggcgtcgagaacttcttcgcccactaccagaagggcttcaaggagaaggtggagctggcggagatgaagaacgtcctcaacagcaacctgaagaacggcaacctcgagcacgtcttctgcccgatcaggggcggcaggtactacctgtcgacgcaccccgtcacgagcgacgctaagcccttcgagttcaacggccgcaagtgctacatctgcgactccgacgaggtggcggccaccaacatcatgctgatcggcctcttctacgtgtga

SEQ ID NO: 6 - DNA, 3165 bp, synthetic construct
  misc_feature 1..3165: note = encoding nucleic acid sequence of Cas12f.6
  source 1..3165: mol_type = other DNA
atgtccagcgccatcaagtcctacaagagcgtgctgcgccccaacgagaggaagaaccagctcctgaagtccaccatccagtgcctcgaggacggcagcgccttcttcttcaagatgctgcagggcctcttcggtggcatcaccccggagatcgtcaggttctccacggagcaggagaaacagcagcaggacatcgccctgtggtgcgccgtcaactggttcaggcccgtgtcccaggacagcctcacccacacgatcgccagcgacaacctggtggagaagttcgaggagtactacggcggcacggcctccgacgcgatcaagcagtacttctccgcgagcatcggcgagagctactactggaacgactgcaggcagcagtactacgacctgtgcagggagctgggcgtggaggtctccgacctgacccacgacctggagatcctgtgcagggagaagtgcctggcggtggccaccgagagcaaccagaacaactccatcatcagcgtcctgttcggcaccggcgagaaggaggacaggtccgtgaagctcaggatcacgaagaagatcctcgaggccatcagcaacctgaaggagatcccgaagaacgtcgcgcccatccaggagatcatcctgaacgtggccaaggcgaccaaggagacgttcaggcaggtgtacgcgggcaacctcggcgccccatccaccctggagaagttcatcgccaaggacggccagaaggagttcgacctgaagaagctccagacggacctcaagaaggtcatccgcggcaagtccaaggagagggactggtgctgccaggaggagctgcgcagctacgtggagcagaacaccatccagtacgacctctgggcctggggcgagatgttcaacaaggcccacaccgcgctcaagatcaagtccacgaggaactacaacttcgcgaagcagcgcctcgagcagttcaaggagatccagagcctgaacaacctcctggtggtcaagaagctcaacgacttcttcgactccgagttcttcagcggcgaggagacctacacgatctgcgtgcaccacctgggcggcaaggacctgtccaagctctacaaggcctgggaggacgaccccgccgaccccgagaacgcgatcgtggtcctgtgcgacgacctcaagaacaacttcaagaaggagccgatccgcaacatcctcaggtacatcttcaccatcaggcaggagtgctcggctcaggacatcctggcggccgccaagtacaaccagcagctcgaccgctacaagtcccagaaggccaacccatcggtcctgggcaaccagggcttcacctggacgaacgccgtgatcctgcccgagaaggcccagaggaacgacaggcccaactccctcgacctgaggatctggctctacctgaagctcaggcaccccgacggcaggtggaagaagcaccacatccccttctacgacacccgcttcttccaggagatctacgccgcgggcaacagccccgtggacacctgccagttccgcacgccgaggttcggctaccacctgcccaagctcaccgaccagacggccatcagggtcaacaagaagcacgtcaaggccgctaagacggaggctcgcatcaggctggcgatccagcagggcacgctcccggtgtccaacctgaagatcaccgagatctccgcgacgatcaacagcaagggccaggtgcgcatcccggtcaagttcgacgtgggcaggcagaagggcaccctccagatcggcgaccgcttctgcggctacgaccagaaccagaccgcctcccacgcctacagcctgtgggaggtggtcaaggagggccagtaccacaaggagctgggctgcttcgtcaggttcatctccagcggcgacatcgtgtccatcaccgagaaccgcggcaaccagttcgaccagctcagctacgagggcctggcctacccccagtacgccgactggaggaagaaggcctccaagttcgtgagcctgtggcagatcaccaagaagaacaagaagaaggagatcgtgacggtcgaggccaaggagaagttcgacgcgatctgcaagtaccagccgcgcctctacaagttcaacaaggagtacgcctacctcctgcgcgacatcgtcaggggcaagagcctggtggagctgcagcagatccgccaggagatcttcaggttcatcgagcaggactgcggcgtcacgcgcctgggctccctgagcctctccaccctggagacggtgaaggccgtcaagggcatcatctactcctacttcagcacggccctgaacgcgtccaagaacaacccgatcagcgacgagcagcgcaaggagttcgaccccgagctgttcgccctcctggagaagctggagctgatccgcacccgcaagaagaagcagaaggtggagaggatcgcgaactccctcatccagacgtgcctggagaacaacatcaagttcatccgcggcgagggcgacctgagcaccacgaacaacgccaccaagaagaaggcgaacagccgcagcatggactggctggccaggggcgtcttcaacaagatccgccagctcgcgccgatgcacaacatcaccctcttcggctgcggctccctgtacacgagccaccaggacccgctcgtgcacaggaaccccgacaaggccatgaagtgcaggtgggccgctatcccggtcaaggacatcggcgactgggtgctgaggaagctctcccagaacctgcgcgcgaagaacatcggcacgggcgagtactaccaccagggcgtcaaggagttcctcagccactacgagctgcaggacctcgaggaggagctgctgaagtggcgctccgacaggaagagcaacatcccctgctgggtgctgcagaaccgcctcgccgagaagctgggcaacaaggaggccgtggtctacatccccgtccgcggcggcaggatctacttcgctacccacaaggtggctaccggcgcggtgtccatcgtcttcgaccagaagcaagtgtgggtctgcaacgcggaccacgtcgccgctgccaacatcgccctgaccgtgaagggcatcggcgagcagtccagcgacgaggagaacccggacggcagcaggatcaagctgcagctcaccagctga

SEQ ID NO: 7 - RNA, 63 nt, synthetic construct
  misc_feature 1..63: note = Cas12f.4/prototype direct repeat
  source 1..63: mol_type = other RNA
ctctgaccacctgagagaatgtgtgcatagtcacacggtataacaacttcgacgagctctaca

SEQ ID NO: 8 - RNA, 63 nt, synthetic construct
  misc_feature 1..63: note = Cas12f.5/prototype direct repeat
  source 1..63: mol_type = other RNA
ataacaatagatagaaaatgtgtcatactacgacacggtataacaacttcgacgagctctaca

SEQ ID NO: 9 - RNA, 54 nt, synthetic construct
  misc_feature 1..54: note = Cas12f.6/prototype direct repeat
  source 1..54: mol_type = other RNA
cctaagaaatccgtctttcattgacggggtataacaacttcgacgagctctaca

SEQ ID NO: 10 - DNA, 63 bp, synthetic construct
  misc_feature 1..63: note = encoding nucleic acid sequence of Cas12f.4/prototype direct repeat
  source 1..63: mol_type = other DNA
ctctgaccacctgagagaatgtgtgcatagtcacacggtataacaacttcgacgagctctaca

SEQ ID NO: 11 - DNA, 63 bp, synthetic construct
  misc_feature 1..63: note = encoding nucleic acid sequence of Cas12f.5/prototype direct repeat
  source 1..63: mol_type = other DNA
ataacaatagatagaaaatgtgtcatactacgacacggtataacaacttcgacgagctctaca

SEQ ID NO: 12 - DNA, 54 bp, synthetic construct
  misc_feature 1..54: note = encoding nucleic acid sequence of Cas12f.6/prototype direct repeat
  source 1..54: mol_type = other DNA
cctaagaaatccgtctttcattgacggggtataacaacttcgacgagctctaca

SEQ ID NO: 13 - RNA, 23 nt, synthetic construct
  misc_feature 1..23: note = Cas12f.4/mature direct repeat
  source 1..23: mol_type = other RNA
agagaatgtgtgcatagtcacac

SEQ ID NO: 14 - RNA, 24 nt, synthetic construct
  misc_feature 1..24: note = Cas12f.5/mature direct repeat
  source 1..24: mol_type = other RNA
agaaaatgtgtcatactacgacac

SEQ ID NO: 15 - RNA, 23 nt, synthetic construct
  misc_feature 1..23: note = Cas12f.6/mature direct repeat
  source 1..23: mol_type = other RNA
agaaatccgtctttcattgacgg

SEQ ID NO: 16 - DNA, 23 bp, synthetic construct
  misc_feature 1..23: note = encoding nucleic acid sequence of Cas12f.4/mature direct repeat
  source 1..23: mol_type = other DNA
agagaatgtgtgcatagtcacac

SEQ ID NO: 17 - DNA, 24 bp, synthetic construct
  misc_feature 1..24: note = encoding nucleic acid sequence of Cas12f.5/mature direct repeat
  source 1..24: mol_type = other DNA
agaaaatgtgtcatactacgacac

SEQ ID NO: 18 - DNA, 23 bp, synthetic construct
  misc_feature 1..23: note = encoding nucleic acid sequence of Cas12f.6/mature direct repeat
  source 1..23: mol_type = other DNA
agaaatccgtctttcattgacgg

SEQ ID NO: 19 - protein, 11 aa, synthetic construct
  REGION 1..11: note = NLS sequence
SRADPKKKRKV

SEQ ID NO: 20 - protein, 1056 aa, synthetic construct
  REGION 1..1056: note = amino acid sequence of Cas12f.4-NLS fusion protein
MKKVEVSRPYQSLLLPNHRKFKYLDETWNAYKSVKSLLHRFLVCAYGAVPFNKFVEVVEKVDNDQLVLAFAVRLFRLVPVESTSFAKVDKANLAKSLANHLPVGTAIPANVQSYFDSNFDPKKYMWIDCAWEADRLAREMGLSASQFSEYATTMLWEDWLPLNKDDVNGWGSVSGLFGEGKKEDRQQKVKMLNNLLNGIKKNPPKDYTQYLKILLNAFDAKSHKEAVKNYKGDSTGRTASYLSEKSGEITELMLEQLMSNIQRDIGDKQKEISLPKKDVVKKYLESESGVPYDQNLWSQAYRNAASSIKKTDTRNFNSTLEKFKNEVELRGLLSEGDDVEILRSKFFSSEFHKTPDKFVIKPEHIGFNNKYNVVAELYKLKAEATDFESAFATVKDEFEEKGIKHPIKNILEYIWNNEVPVEKWGRVARFNQSEEKLLRIKANPTVECNQGMTFGNSAMVGEVLRSNYVSKKGALVSGEHGGRLIGQNNMIWLEMRLLNKGKWETHHVPTHNMKFFEEVHAYNPSLADSVNVRNRLYRSEDYTQLPSSITDGLKGNPKAKLLKRQHCALNNMTANVLNPKLSFTINKKNDDYTVIIVHSVEVSKPRREVLVGDYLVGMDQNQTASNTYAVMQVVKPKSTDAIPFRNMWVRFVESGSIESRTLNSRGEYVDQLNHDGVDLFEIGDTEWVDSARKFFNKLGVKHKDGTLVDLSTAPRKAYAFNNFYFKTMLNHLRSNEVDLTLLRNEILRVANGRFSPMRLGSLSWTTLKALGSFKSLVLSYFDRLGAKEMVDKEAKDKSLFDLLVAINNKRSNKREERTSRIASSLMTVAQKYKVDNAVVHVVVEGNLSSTDRSASKAHNRNTMDWCSRAVVKKLEDMCNLYGFNIKGVPAFYTSHQDPLVHRADYDDPKPALRCRYSSYSRADFSKWGQNALAAVVRWASNKKSNTCYKVGAVEFLKQHGLFADKKLTVEQFLSKVKDEEILIPRRGGRVFLTTHRLLAESTFVYLNGVKYHSCNADEVAAVNICLNDWVIPCKKKMKEESSASGSRADPKKKRKV

SEQ ID NO: 21 - protein, 1090 aa, synthetic construct
  REGION 1..1090: note = amino acid sequence of Cas12f.5-NLS fusion protein
MPKQKDLPYSCLSYLQPNERKLKLLNNTYDHLTHGSKIMFDTLIALMGGINPKMDVISENKDSEIKNNRDPQTMCATIWFRPMKSKRINKVWSPKQLKEQFLKYYQEYEADVKINDMVEAYFDSPLGENYVWVDCRKKYKQLVKELASIAKTTEANLKEDLDCDLECLFRPSEKKMKLYGSNKSWAIISNLFGEGDKEDRSKKIKILTKAIQILTESNPESYADVQKAFLAAADIDDPKKFHTQEIWGNGSPGNIVKMARGDFLGKEFDCEKILEKINDVLKEKTLDFDLKVRLSFKEYLISKIGHYYQNSWSEMINSAFADIISKNTRNVNFAKEKVQLQKTLSETSNAKVELLTDFFKSDFFLGDDKFDIAPHNLGGANGIKFFYDFCKKNEDQYFLEELLLEAAIEESVAEAKSKSLKEPHKDLLRYVFSIRKETTFEELRDAAKYIQTHKRIKNMSVHPTVKSDIGFNVTSGSALVGHVVSPSKKINGRIAGESGFIWICMKLWEGGDKWIEHHIPFTDTRFYEQIYKYNPDSKLEPVVLRTKRYGVDLTKFNLPPMKTDLKHVAPKEKNKHNYVKVQRRLQRLNHPDVPNTIWPKSNIGFTIRRKNGKYILNVVHKLPKNKVKKSVKPKFGDILIGVDQNQTTNHTCSIYKVVKKNTKEALLVPESDFYLKKIETIKVTSFTKARYNSEPIDQLHYEGISVDNEVFKNWCKDREQFVDSLSIKEFKNEFKRIKNKNENLYSFNADYLWLLKRIISGKLNKKKFDVSVFEKSIRNEILAMCSKEGLGPLRVSSLSSNSLKSIGFLKSAICSFISIALNRKGIEDKTDVQKNKIDPELFDLIGKIEQKRVNKRMEKTRRNADFILTMAVDYQKSSQKNVFLFCEGNLETAKTGNSKKRNSANVDWCSRKLFDFLKEKSLRHGIYFHAVTPHYTSHQDPFEYHPSNKVMLPRFAKFDKNNPIQDWAEKKYLGFANSDPESGTALYYKKGVENFFAHYQKGFKEKVELAEMKNVLNSNLKNGNLEHVFCPIRGGRYYLSTHPVTSDAKPFEFNGRKCYICDSDEVAATNIMLIGLFYVSRADPKKKRKV

SEQ ID NO: 22 - protein, 1065 aa, synthetic construct
  REGION 1..1065: note = amino acid sequence of Cas12f.6-NLS fusion protein
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTSSRADPKKKRKV

SEQ ID NO: 23 - DNA, 3713 bp, synthetic construct
  misc_feature 1..3713: note = Plasmid expressing Cas12f.4 system
  source 1..3713: mol_type = other DNA
tttacactttatgcttccggctcgtatgttaggaggtctttatcatgtctaacaaagaaaaaaatgcaagcgaaactcgcaaagcctacacaacaaaaatgattccaagaagccatgatcgcatgaaattgcttgggaatttcatggattatttgatggatggaacgccaatatttttcgaactttggaatcagtttggcggcgggattgaccgcgatatcatttctggcactgcaaataaagacaagatatcagatgatttacttttggcggtcaattggttcaaggtaatgccaattaattctaagcctcaaggtgtatcgccatcaaatcttgccaacctctttcaacaatactctggatcagaaccagacattcaagctcaagagtattttgcttcaaattttgacaccgaaaagcatcaatggaaggacatgcgtgttgaatacgaacgactattagctgaattgcagctatcgagaagtgatatgcatcatgacttgaagctcatgtacaaagaaaaatgcattggcctaagtctttctacggctcactacatcacttctgtgatgtttgggacaggagctaaaaacaatcgccaaaccaagcatcaattctatagcaaggttatccaactacttgaggaatcaactcaaatcaattctgttgaacagttggcatctattattttgaaagcaggagattgcgatagttatcgaaagcttcgtattcgatgttctcgtaagggagcaacacccagcattcttaagatcgttcaagactatgaactgggaaccaatcacgatgatgaagtgaatgtgccaagtttgattgcaaatttgaaagaaaaattgggcagatttgaatatgaatgcgaatggaagtgcatggaaaaaatcaaagcatttttagctagcaaagttgggccttattacctaggctcttacagtgcgatgcttgaaaatgcattgtcgcccatcaagggaatgactacaaaaaattgcaaatttgtgttaaagcaaattgatgccaaaaacgacatcaagtatgaaaatgagccatttggcaaaattgttgaagggttttttgactctccatattttgaaagcgacaccaatgtgaaatgggttttgcacccacatcatattggagaaagcaatatcaaaacactctgggaagacttgaatgcaattcattctaagtacgaagaagatattgcttctttgagcgaagacaaaaaagagaaacgcattaaggtttatcaaggagatgtttgccaaacaatcaatacgtattgtgaagaagtaggaaaggaagctaagactcctttagttcagcttttgcgttatctttactctagaaaagatgatattgctgttgataagataattgatggcattaccttccttagcaagaaacacaaggttgaaaaacaaaaaatcaatcctgtaattcaaaaatatcccagtttcaactttgggaataattctaagttgttgggaaagattatcagccccaaagacaagttaaagcataatctcaaatgcaacaggaatcaggttgataattacatttggattgagattaaagtactaaacaccaaaacgatgcgatgggaaaagcatcactatgctttatcatctacgcggtttttggaagaggtctattatccagccacatccgaaaatccgccagacgctttggcagcacgtttccgaactaaaactaatgggtatgaaggcaagcctgcgttgtctgctgagcaaattgaacaaattagatcagccccagtcggtttgagaaaagtgaaaaaacgtcaaatgcgactcgaagctgcaagacagcaaaatctcttgcctcgatacacttggggcaaagatttcaacataaacatttgtaagcgtggcaacaattttgaagtcactcttgcgacgaaggtgaaaaagaaaaaagaaaagaattataaggttgttttagggtacgatgctaatatcgttcgcaaaaacacttacgcagccatagaagctcacgctaatggcgatggtgtgattgactacaatgacttgcccgtgaagcctattgaaagtggatttgtaaccgttgaaagtcaagtgcgagacaaatcttacgatcaactctcttacaatggcgtaaagctcttgtattgcaagcctcatgttgagtctcgacgttcatttttggagaaataccgaaatggcaccatgaaggacaacagaggaaacaacattcaaattgactttatgaaagactttgaagctattgcggatgatgaaacttctttgtattacttcaatatgaagtactgcaagctgcttcaatcgtccattcgcaatcattcttcacaagcaaaagaatatcgtgaagagatttttgaattgttaagagacggaaaactatcggttttgaagttatcatctttgagcaatctttcttttgtgatgttcaaagttgccaaatctctgatcggtacttactttggccacttgcttaagaagccgaagaattctaagtcagatgttaaggcaccgcctataactgatgaagataagcaaaaagctgatcctgagatgtttgctttgaggttggctttggaggagaagcgactaaacaaagtcaagtctaagaaagaagtaattgcgaacaagattgttgctaaggcacttgagcttcgcgacaagtacgggcctgtgttgattaagggagaaaacatctctgacacgaccaagaaaggcaagaagtcaagcaccaattcttttttgatggactggctagcacgcggtgtggctaataaagtcaaagaaatggtaatgatgcatcaaggacttgaatttgtagaagtaaatcctaatttcacatctcaccaagatccttttgttcacaagaaccctgaaaatacgtttagagctaggtacagtcggtgcactccaagtgaacttactgagaaaaatcgcaaggaaattttgagctttttgagcgataagccttctaaacgaccgacaaatgcctattacaatgaaggtgcgatggcctttcttgcaacttatggcttgaagaagaatgatgtgctaggagttagtcttgagaaattcaagcaaataatggccaacattctacatcagcgttccgaagatcaattattgtttccttctagaggtggcatgttttatcttgcaacttacaagcttgatgctgacgctacctctgtaaattggaatggcaaacagttttgggtttgtaacgcagatttagtagcggcatacaatgtcggtttggtcgatattcaaaaagacttcaagaaaaagtaaaaataaaacgaaaggctcagtcgaaagactgggcctttcgttttatctgttgtttgtcggtgaacgctctcctgagtaggacaaatttgacagctagctcagtcctaggtataatgctagcgctgacgttggaatgactaatttttgtgcccaccgttggcacggtataacaacttcgacgagctctacacgttggaatgactaatttttgtgcccaccgttggcacggatcgctgagaccgcatcaaagcacgatgagcgtggcgttggaatgactaatttttgtgcccaccgttggcacaaataaaacgaaaggctcagtcgaaagactgggcctttcgttttatctgttgtttgtcggtgaacgctctcctgagtaggacaaat

SEQ ID NO: 24 - DNA, 35 bp, synthetic construct
  misc_feature 1..35: note = PAM library sequence
  misc_feature 1..8: note = n = a or g or c or t
  source 1..35: mol_type = other DNA
nnnnnnnnggtataacaacttcgacgagctctaca

SEQ ID NO: 25 - RNA, 43 nt, synthetic construct
  misc_feature 1..43: note = Guide RNA-VEGFA of Cas12f.4 system
  source 1..43: mol_type = other RNA
agagaatgtgtgcatagtcacacctaggaatattgaagggggc

SEQ ID NO: 26 - RNA, 44 nt, synthetic construct
  misc_feature 1..44: note = Guide RNA-VEGFA of Cas12f.5 system
  source 1..44: mol_type = other RNA
agaaaatgtgtcatactacgacacctaggaatattgaagggggc

SEQ ID NO: 27 - RNA, 43 nt, synthetic construct
  misc_feature 1..43: note = Guide RNA-VEGFA of Cas12f.6 system
  source 1..43: mol_type = other RNA
agaaatccgtctttcattgacggctaggaatattgaagggggc

SEQ ID NO: 28 - RNA, 44 nt, synthetic construct
  misc_feature 1..44: note = Guide RNA-PDI1 of Cas12f.4 system
  source 1..44: mol_type = other RNA
agagaatgtgtgcatagtcacacccttgtccttgaattcctccg

SEQ ID NO: 29 - RNA, 44 nt, synthetic construct
  misc_feature 1..44: note = Guide RNA-SBE2.2 of Cas12f.4 system
  source 1..44: mol_type = other RNA
agagaatgtgtgcatagtcacaccggtggatttagtcggcttga

SEQ ID NO: 30 - DNA, 40 bp, synthetic construct
  misc_feature 1..40: note = PAM-VEGFA Target
  source 1..40: mol_type = other DNA
tccctctttgctaggaatattgaagggggcaggggaaggc

SEQ ID NO: 31 - DNA, 40 bp, synthetic construct
  misc_feature 1..40: note = PAM-VEGFA Target
  source 1..40: mol_type = other DNA
gccttcccctgcccccttcaatattcctagcaaagaggga

SEQ ID NO: 32 - RNA, 43 nt, synthetic construct; identical to SEQ ID NO: 25
  source 1..43: mol_type = other RNA
  misc_feature 1..43: note = crRNA Cas12f.4

SEQ ID NO: 33 - DNA, 32 bp, synthetic construct
  misc_feature 1..32: note = result of cleavage activity PAM-VEGFA Target
  source 1..32: mol_type = other DNA
tccctctttgctaggaatattgaggggaaggc

SEQ ID NO: 34 - DNA, 31 bp, synthetic construct
  misc_feature 1..31: note = result of cleavage activity PAM-VEGFA Target
  source 1..31: mol_type = other DNA
tccctctttgctaggaatattgaaggaaggc

SEQ ID NO: 35 - DNA, 32 bp, synthetic construct
  misc_feature 1..32: note = result of cleavage activity PAM-VEGFA Target
  source 1..32: mol_type = other DNA
tccctctttgctaggaatattgaagggaaggc

SEQ ID NO: 36 - DNA, 39 bp, synthetic construct
  misc_feature 1..39: note = result of cleavage activity PAM-VEGFA Target
  source 1..39: mol_type = other DNA
tccctctttgctaggaatattgaagggggcagggaaggc

SEQ ID NO: 37 - DNA, 25 bp, synthetic construct
  misc_feature 1..25: note = result of cleavage activity PAM-VEGFA Target
  source 1..25: mol_type = other DNA
tccctctttgctaggaatagaaggc

SEQ ID NO: 38 - DNA, 47 bp, synthetic construct
  misc_feature 1..47: note = PDI1 target
  source 1..47: mol_type = other DNA
cttgatttccttgtccttgaattcctccgcggcagcagagtaggcag

SEQ ID NO: 39 - DNA, 35 bp, synthetic construct
  misc_feature 1..35: note = result of cleavage activity PDI1 target
  source 1..35: mol_type = other DNA
cttgatttccttgtccttgacagcagagtaggcag

SEQ ID NO: 40 - DNA, 38 bp, synthetic construct
  misc_feature 1..38: note = result of cleavage activity PDI1 target
  source 1..38: mol_type = other DNA
cttgatttccttgtccttgaattcagcagagtaggcag

SEQ ID NO: 41 - DNA, 41 bp, synthetic construct
  misc_feature 1..41: note = result of cleavage activity PDI1 target
  source 1..41: mol_type = other DNA
cttgatttccttgtccttgaattcggcagcagagtaggcag

SEQ ID NO: 42 - DNA, 44 bp, synthetic construct
  misc_feature 1..44: note = result of cleavage activity PDI1 target
  source 1..44: mol_type = other DNA
cttgatttccttgtccttgaattccgcggcagcagagtaggcag

SEQ ID NO: 43 - DNA, 32 bp, synthetic construct
  misc_feature 1..32: note = result of cleavage activity PDI1 target
  source 1..32: mol_type = other DNA
cttgatttccttgtccttgaaagagtaggcag

SEQ ID NO: 44 - DNA, 31 bp, synthetic construct
  misc_feature 1..31: note = result of cleavage activity PDI1 target
  source 1..31: mol_type = other DNA
cttgatttccttgtccttgaagagtaggcag

SEQ ID NO: 45 - DNA, 27 bp, synthetic construct
  misc_feature 1..27: note = result of cleavage activity PDI1 target
  source 1..27: mol_type = other DNA
cttgatttccttgtgcagagtaggcag

SEQ ID NO: 46 - DNA, 34 bp, synthetic construct
  misc_feature 1..34: note = result of cleavage activity PDI1 target
  source 1..34: mol_type = other DNA
cttgatttccttgtccttgaagcagagtaggcag

SEQ ID NO: 47 - DNA, 29 bp, synthetic construct
  misc_feature 1..29: note = result of cleavage activity PDI1 target
  source 1..29: mol_type = other DNA
cttgatttccttgtccttagagtaggcag

SEQ ID NO: 48 - DNA, 47 bp, synthetic construct
  misc_feature 1..47: note = SBE2.2 target
  source 1..47: mol_type = other DNA
ggccttttcggtggatttagtcggcttgatcatgatgccgagtactt

SEQ ID NO: 49 - DNA, 36 bp, synthetic construct
  misc_feature 1..36: note = result of cleavage activity SBE2.2 target
  source 1..36: mol_type = other DNA
ggccttttcggtggatttagtcgatgccgagtactt

SEQ ID NO: 50 - DNA, 36 bp, synthetic construct; identical to SEQ ID NO: 49
  misc_feature 1..36: note = result of cleavage activity SBE2.2 target
  source 1..36: mol_type = other DNA

SEQ ID NO: 51 - DNA, 35 bp, synthetic construct
  misc_feature 1..35: note = result of cleavage activity SBE2.2 target
  source 1..35: mol_type = other DNA
ggccttttcggtggatttaatgatgccgagtactt

SEQ ID NO: 52 - DNA, 35 bp, synthetic construct
  misc_feature 1..35: note = result of cleavage activity SBE2.2 target
  source 1..35: mol_type = other DNA
ggccttttcggtggatttagtcatgccgagtactt

SEQ ID NO: 53 - DNA, 33 bp, synthetic construct
  misc_feature 1..33: note = result of cleavage activity SBE2.2 target
  source 1..33: mol_type = other DNA
ggccttttcggtggatttaaatgccgagtactt

SEQ ID NO: 54 - DNA, 29 bp, synthetic construct
  misc_feature 1..29: note = result of cleavage activity SBE2.2 target
  source 1..29: mol_type = other DNA
ggccttttcggtggatttgccgagtactt